(19) United States
(12) Patent Application Publication
(10) Pub. No.: US 2024/0350607 A1
(43) Pub. Date: Oct. 24, 2024

(54) TUBERCULOSIS VACCINES

(71) Applicant: Vir Biotechnology, Inc., San Francisco, CA (US)

(72) Inventors: Ann M. Arvin, San Francisco, CA (US); Julia Di Iulio, San Francisco, CA (US); Janet L. Douglas, San Francisco, CA (US); Emily Marshall, San Francisco, CA (US); Leah B. Soriaga, San Francisco, CA (US); Herbert W. Virgin, San Francisco, CA (US)

(21) Appl. No.: 18/687,463

(22) PCT Filed: Aug. 30, 2022
(86) PCT No.: PCT/US2022/075645

§ 371 Date: Feb. 28, 2024

Related U.S. Application Data
(60) Provisional application No. 63/392,778, filed on Jul. 27, 2022.
(60) Provisional application No. 63/239,278, filed on Aug. 31, 2021.

(51) Int. Cl. A61K 39/04; A61K 39/00; C07K 14/35; C12N 5/0783; C12N 15/86

(52) U.S. Cl. CPC A61K 39/04; A61K 39/4611; C07K 14/35; C12N 5/0636; C12N 15/86; A61K 2039/5256; C07K 2319/42; C12N 2510/00; C12N 2710/16143

(57) ABSTRACT

The disclosure relates to tuberculosis antigens and vectors for delivering the antigens. The disclosure also relates to immunogenic compositions comprising the same, and their uses.

### STATEMENT REGARDING SEQUENCE LISTING

The Sequence Listing associated with this application is provided in XML format in lieu of a paper copy, and is hereby incorporated by reference into the specification. The name of the XML file containing the Sequence Listing is 930485_439WO_SequenceListing.xml. The XML file is 558,770 bytes, was created on Aug. 22, 2022, and is being submitted electronically via EFS-Web.

### BACKGROUND

Tuberculosis remains a leading cause of disease and mortality globally (Schito, M et al. Perspectives on Advances in Tuberculosis Diagnostics, Drugs, and Vaccines. Clin Infect Dis. 61 Suppl 3, S102-118 (2015)). Accordingly, there remains a need for effective preventative or therapeutic vaccines for Mycobacterium tuberculosis infections.

Cytomegalovirus (CMV)-based vaccine vectors have been found to result in strong immune responses to delivered antigens, even for pathogens that have traditionally been able to evade natural immunity and cause repeated or chronic infection. For example, the 68-1 strain of rhesus cytomegalovirus (RhCMV) modified to encode simian immunodeficiency virus (SIV) antigens has been associated with long-lasting protection against SIV challenge (Hansen, S G et al., Immune clearance of highly pathogenic SIV infection. Nature 502, 100-104 (2013); Hansen, S G et al., Profound early control of highly pathogenic SIV by an effector memory T-cell vaccine. Nature 473, 523-527 (2011); Hansen, S G et al., Effector memory T cell responses are associated with protection of rhesus monkeys from mucosal simian immunodeficiency virus challenge. Nat Med. 15, 293-299 (2009)). Subsequent research with CMV vectors revealed that different immune responses could be elicited depending on the specific genetic components of the CMV backbone (Fruh, K et al., CD8+ T cell programming by cytomegalovirus vectors: applications in prophylactic and therapeutic vaccination. Curr Opin Immunol. 47, 52-56 (2017); Hansen, S G et al. Cytomegalovirus vectors violate CD8+ T cell epitope recognition paradigms. Science 340, 1237874 (2013)). The 68-1 of Rhesus cytomegalovirus (RhCMV) has been shown to elicit CD8+ T cells that recognize peptides presented by MHC-II and MHC-E instead of conventional MIIC-I. This effect has also been observed in cynomolgus monkey CMV (CyCMV), demonstrating that deletion of the RhCMV and CyCMV homologs of HCMV UL128, UL130, UL146, and UL147 enables the induction of MHC-E-restricted CD8+ T cells (International Application Publication Nos. WO2016/130693A1, WO2018/075591A1). In addition, these vectors elicit MHC-II restricted CD8+ T cells. The induction of MHC-II restricted CD8+ T cells can be eliminated by the insertion of a targeting site for the endothelial cell specific micro RNA (miR) 126 into essential viral genes of these vectors, resulting in “MHC-E only” vectors that exclusively elicit MHC-E restricted CD8+ T cells (International Application Publication No. WO2018/075591A1). In contrast, insertion of the myeloid cell specific miR142-3p into 68-1 RhCMV has been shown to prevent the induction of MHC-E restricted CD8+ T cells, resulting in vectors that elicit CD8+ T cells exclusively restricted by MHC-II (International Application Publication No. WO2017/087921A1). Deletion of the UL40 homolog Rh67 has also been shown to prevent the induction of MHC-E restricted CD8+ T cells, resulting in “MHC-II-only vectors” (International Application Publication No. WO2016/130693A1). Accordingly, by designing CMV vectors to have particular gene deletions, CMV can be used to deliver antigens and “program” immune responses to those antigens.

### BRIEF SUMMARY

Disclosed herein are fusion proteins comprising Mycobacterium tuberculosis (Mtb) antigens and nucleic acids encoding the fusion proteins. In some embodiments, the present disclosure provides a fusion protein comprising one or more of Mtb Ag85A, ESAT-6, Rv3407, Rv2626c, RpfA, RpfD, Ra12, TbH9, and Ra35, or portions or fragments thereof. In some embodiments, the present disclosure provides vectors encoding a fusion protein as described above.

### BRIEF DESCRIPTION OF THE DRAWING

FIG. 1 shows examples of fusion proteins comprising Mtb antigens.

FIG. 2 shows fusion proteins Fusion 6, Fusion 7, and Fusion 8 comprising Mtb antigens. “M72” in the figure refers to M72-fusion-2.

FIG. 3 summarizes protein conservation of Mtb antigens and RpfA variants. A total of 4884 strains/isolates were included in analysis across all Mtb antigens and RpfA variants. The number of aligned isolates per amino acid position is indicated in the figure. Isolates annotated as “Rv0867c” were included in the analysis of RpfA variants.

FIG. 4 shows variable RpfA protein length distributed across Mtb isolates. Circled areas indicate groups of isolates with different categories of truncations and/or deletions.

FIG. 5 shows geographical distribution of full-length RpfA variants. The analysis was performed on the group of isolates labeled “Isolates with the full-length protein” from FIG. 4. The top twenty geographical locations with the largest fraction of isolates bearing full-length RpfA genes are shown. Full-length RpfA was defined as longer than 400 amino acids. The y-axis label “missing” refers to isolates with unknown location information.

FIGS. 6A-6L show frequencies of “responding” (cytokine-expressing) CD4+ or CD8+ T cells (as indicated) in peripheral blood mononuclear cells (PBMCs) isolated from rhesus macaques administered viral vectors expressing Fusion 6 under the control of either a UL78 or UL82 promoter. PBMCs were collected at 0, 2, 4, 6, 8, and 10 weeks post-dosing and stimulated with Mtb peptide pools containing peptides from genes expressed in Fusion 6 (Ag58A (FIGS. 6A-6B), Rv2626 (FIGS. 6C-6D), RpfA (FIGS. 6E-6F), ESAT6 (FIGS. 6G-6H), Rv3407 (FIGS. 6I-6J), or RpfD (FIGS. 6K-6L)) prior to intracellular cytokine staining (ICS). Solid lines indicate a viral vector dose of 10⁶ pfu, dotted lines indicate a viral vector dose of 10⁵ pfu.

FIGS. 7A-7N show frequencies of “responding” (cytokine-expressing) CD4+ or CD8+ T cells (as indicated) in peripheral blood mononuclear cells (PBMCs) isolated from rhesus macaques administered viral vectors expressing Fusion 7 under the control of either a UL78 or UL82 promoter or Fusion 8 under the control of a UL82 promoter. PBMCs were collected at 0, 2, 4, and 6 weeks post-dosing and stimulated with Mtb peptide pools containing peptides from genes expressed in Fusion 6 (Ag58A (FIGS. 7A-7B), Rv2626 (FIGS. 7C-7D), RpfA (FIGS. 7E-7F), ESAT6 (FIGS. 7G-7H), Rv3407 (FIGS. 7I-7J), or RpfD (FIGS. 7K-7L)) and M72-fusion-2 (labeled “M72” in FIGS. 7M-7N) prior to intracellular cytokine staining (ICS). Solid lines indicate a viral vector dose of 10⁶ pfu, dotted lines indicate a viral vector dose of 10⁵ pfu.

FIG. 8 summarizes potential indications for Vector 4 (SEQ ID NO:44). “TB” refers to tuberculosis. “NHP” refers to non-human primates.

FIG. 9 shows a development plan to evaluate Vector 4 (SEQ ID NO:44) for use in prevention of pulmonary tuberculosis in adolescents and adults

FIG. 10 shows a development plan to evaluate Vector 4 (SEQ ID NO:44) for use in prevention of Mtb infection and prevention of tuberculosis relapse in adolescents and adults.

### DETAILED DESCRIPTION

### I. Glossary

The following sections provide a detailed description of tuberculosis antigens, and related pharmaceutical compositions and methods of inducing an immune response, such as an anti-Mycobacterium tuberculosis response, and methods of treating or preventing tuberculosis. Prior to setting forth this disclosure in more detail, it may be helpful to an understanding thereof to provide definitions of certain terms to be used herein. Additional definitions are set forth throughout this disclosure.

Unless the context requires otherwise, throughout the present specification and claims, the word “comprise” and variations thereof, such as “comprises” and “comprising,” are to be construed in an open, inclusive sense, that is, as “including, but not limited to”. “Consisting of” shall mean excluding more than trace elements of other ingredients and substantial method steps disclosed herein, and in the case of an amino acid or nucleic acid sequence, excluding additional amino acids or nucleotides, respectively. The term “consisting essentially of” limits the scope of a claim to the specified materials or steps, or to those that do not materially affect the basic characteristics of a claimed invention. For example, a composition consisting essentially of the elements as defined herein would not exclude trace contaminants from the isolation and purification method and pharmaceutically acceptable carriers, such as phosphate buffered saline, preservatives, and the like. Similarly, a protein consists essentially of a particular amino acid sequence when the protein includes additional amino acids that contribute to at most 20% of the length of the protein and do not substantially affect the activity of the protein (e.g., alters the activity of the protein by no more than 50%). Embodiments defined by each of the transitional terms are within the scope of this invention.

In the present description, the term “about” means±20% of the indicated range, value, or structure, unless otherwise indicated.

It should be understood that the terms “a” and “an” as used herein include “one or more” of the enumerated components unless stated otherwise. The use of the alternative (e.g., “or”) should be understood to mean either one, both, or any combination thereof of the alternatives, and may be used synonymously with “and/or”. As used herein, the terms “include” and “have” are used synonymously, which terms and variants thereof are intended to be construed as non-limiting.

The word “substantially” does not exclude “completely”; e.g., a composition which is “substantially free” from Y may be completely free from Y. Where necessary, the word “substantially” may be omitted from definitions provided herein.

As used herein, the terms “peptide”, “polypeptide”, and “protein” and variations of these terms refer to a molecule, in particular a peptide, oligopeptide, polypeptide, or protein including fusion protein, respectively, comprising at least two amino acids joined to each other by a normal peptide bond, or by a modified peptide bond, such as for example in the cases of isosteric peptides. For example, a peptide, polypeptide, or protein may be composed of amino acids selected from the 20 amino acids defined by the genetic code, linked to each other by a normal peptide bond (“classical” polypeptide). A peptide, polypeptide, or protein can be composed of L-amino acids and/or D-amino acids. In particular, the terms “peptide”, “polypeptide”, and “protein” also include “peptidomimetics,” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. In particular, a peptide, polypeptide, or protein may comprise amino acids other than the 20 amino acids defined by the genetic code in addition to these amino acids, or it can be composed of amino acids other than the 20 amino acids defined by the genetic code. In particular, a peptide, polypeptide, or protein in the context of the present disclosure can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain, or even at the carboxy- or amino-terminal ends. In particular, a peptide or polypeptide can be branched following an ubiquitination or be cyclic with or without branching. This type of modification can be the result of natural or synthetic post-translational processes that are well known to a person skilled in the art. The terms “peptide”, “polypeptide”, or “protein” in the context of the present disclosure in particular also include modified peptides, polypeptides, and proteins. For example, peptide, polypeptide, or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation, or ubiquitination. Such modifications are fully detailed in the literature (Proteins Structure and Molecular Properties, 2nd Ed., T. E. Creighton, New York (1993); Post-translational Covalent Modifications of Proteins, B. C. Johnson, Ed., Academic Press, New York (1983); Seifter, et al., Analysis for protein modifications and nonprotein cofactors, Meth. Enzymol. 182:626-46 (1990); and Rattan, et al., Protein Synthesis: Post-translational Modifications and Aging, Ann NY Acad Sci 663:48-62(1992)). Accordingly, the terms “peptide”, “polypeptide”, and “protein” include for example lipopeptides, lipoproteins, glycopeptides, glycoproteins, and the like. “Orthologs” of proteins are typically characterized by possession of greater than 75% sequence identity counted over the full-length alignment with the amino acid sequence of specific protein using an alignment algorithm, for example, the ALIGN program (version 2.0) set to default parameters. Proteins with even greater similarity to a reference sequence will show increasing percentage identities when assessed by this method, such as at least 80%, at least 85%, at least 90%, at least 92%, at least 95%, or at least 98% sequence identity. In addition, sequence identity can be compared over the full length of particular domains of the disclosed peptides.

As used herein a “(poly)peptide” comprises a single chain of amino acid monomers linked by peptide bonds as explained above. A “protein”, as used herein, comprises one or more, e.g., 1, 2, 3, 4, 5, 6, 7, 8, 9 or 10 (poly)peptides, i.e., one or more chains of amino acid monomers linked by peptide bonds as explained above. In particular embodiments, a protein according to the present disclosure comprises 1, 2, 3, or 4 polypeptides.

As used herein, the terms “nucleic acid”, “nucleic acid molecule,” “nucleic acid sequence,” and “polynucleotide” are used interchangeably and are intended to include DNA molecules and RNA molecules, including, without limitation, messenger RNA (mRNA), DNA/RNA hybrids, or synthetic nucleic acids. The nucleic acid may be single-stranded, or partially or completely double stranded (duplex). Duplex nucleic acids may be homoduplex or heteroduplex. A nucleic acid molecule may be single-stranded or double-stranded.

As used herein, the term “coding sequence” is intended to refer to a polynucleotide molecule, which encodes the amino acid sequence of a protein product. The boundaries of the coding sequence are generally determined by an open reading frame, which usually begins with an ATG start codon.

The term “expression” as used herein refers to any step involved in the production of the polypeptide, including transcription, post-transcriptional modification, translation, post-translational modification, secretion, or the like.

As used herein, the term “sequence variant” refers to any sequence having one or more alterations in comparison to a reference sequence, whereby a reference sequence is any of the sequences listed in the sequence listing, i.e., SEQ ID NO:1 to SEQ ID NO:40. Thus, the term “sequence variant” includes nucleotide sequence variants and amino acid sequence variants. For a sequence variant in the context of a nucleotide sequence, the reference sequence is also a nucleotide sequence, whereas for a sequence variant in the context of an amino acid sequence, the reference sequence is also an amino acid sequence. A “sequence variant” as used herein is at least 80%, at least 85%, at least 90%, at least 95%, at least 98%, or at least 99% identical to the reference sequence. Sequence identity is usually calculated with regard to the full length of the reference sequence (i.e., the sequence recited in the application), unless otherwise specified. Percentage identity, as referred to herein, can be determined, for example, using various methods of alignment known in the art, such as BLAST using the default parameters specified by the NCBI (the National Center for Biotechnology Information; http://www.ncbi.nlm.nih.gov/) [Blosum 62 matrix; gap open penalty=1 1 and gap extension penalty=1]. A “sequence variant” in the context of a nucleic acid (nucleotide) sequence has an altered sequence in which one or more of the nucleotides in the reference sequence is deleted, or substituted, or one or more nucleotides are inserted into the sequence of the reference nucleotide sequence. Nucleotides are referred to herein by the standard one-letter designation (A, C, G, or T). Due to the degeneracy of the genetic code, a “sequence variant” of a nucleotide sequence can either result in a change in the respective reference amino acid sequence, i.e., in an amino acid “sequence variant” or not. In certain embodiments, the nucleotide sequence variants are variants that do not result in amino acid sequence variants (i.e., silent mutations). However, nucleotide sequence variants leading to “non-silent” mutations are also within the scope, in particular such nucleotide sequence variants, which result in an amino acid sequence, which is at least 80%, at least 85%, at least 90%, at least 95%, at least 98%, or at least 99% identical to the reference amino acid sequence. A “sequence variant” in the context of an amino acid sequence has an altered sequence in which one or more of the amino acids is deleted, substituted or inserted in comparison to the reference amino acid sequence. As a result of the alterations, such a sequence variant has an amino acid sequence which is at least 80%, at least 85%, at least 90%, at least 95%, at least 98%, or at least 99% identical to the reference amino acid sequence. For example, per 100 amino acids of the reference sequence a variant sequence having no more than 10 alterations, i.e., any combination of deletions, insertions, or substitutions, is “at least 90% identical” to the reference sequence.

While it is possible to have non-conservative amino acid substitutions, in certain embodiments, the substitutions are conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g., alanine, valine, leucine, and isoleucine, with another; substitution of one hydroxyl-containing amino acid, e.g., serine and threonine, with another; substitution of one acidic residue, e.g., glutamic acid or aspartic acid, with another; replacement of one amide-containing residue, e.g., asparagine and glutamine, with another; replacement of one aromatic residue, e.g., phenylalanine and tyrosine, with another; replacement of one basic residue, e.g., lysine, arginine, and histidine, with another; and replacement of one small amino acid, e.g., alanine, serine, threonine, methionine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

Unless otherwise stated, alterations in the sequence variants do not abolish the functionality of the respective reference sequence, for example, in the present case, the functionality of an antigen or vector disclosed herein. Guidance in determining which nucleotides and amino acid residues, respectively, may be substituted, inserted, or deleted without abolishing such functionality can be found by using computer programs well known in the art.

The nucleotide sequences of the present disclosure may be codon optimized, for example the codons may be optimized for use in human cells. For example, any viral or bacterial sequence may be so altered. Many viruses, including HIV and other lentiviruses, use a large number of rare codons and, by altering these codons to correspond to codons commonly used in the desired subject, enhanced expression of antigens may be achieved as described in Andre, S et al. (Increased Immune Response Elicited by DNA Vaccination with a Synthetic gp120 Sequence with Optimized Codon Usage. J Virol. 72, 1497-1503 (1998)).

As used herein, a nucleic acid sequence or an amino acid sequence “derived from” a designated nucleic acid, peptide, polypeptide, or protein refers to the origin of the nucleic acid, peptide, polypeptide, or protein. In some embodiments, the nucleic acid sequence or amino acid sequence which is derived from a particular sequence has an amino acid sequence that is essentially identical to that sequence or a portion thereof, from which it is derived, whereby “essentially identical” includes sequence variants as defined above. In certain embodiments, the nucleic acid sequence or amino acid sequence which is derived from a particular peptide or protein is derived from the corresponding domain in the particular peptide or protein. Thereby, “corresponding” refers in particular to the same functionality. For example, an “extracellular domain” corresponds to another “extracellular domain” (of another protein), or a “transmembrane domain” corresponds to another “transmembrane domain” (of another protein). “Corresponding” parts of peptides, proteins, and nucleic acids are thus identifiable to one of ordinary skill in the art. Likewise, sequences “derived from” other sequences are usually identifiable to one of ordinary skill in the art as having its origin in the sequence.

In some embodiments, a nucleic acid sequence or an amino acid sequence derived from another nucleic acid, peptide, polypeptide, or protein may be identical to the starting nucleic acid, peptide, polypeptide, or protein (from which it is derived). However, a nucleic acid sequence or an amino acid sequence derived from another nucleic acid, peptide, polypeptide, or protein may also have one or more mutations relative to the starting nucleic acid, peptide, polypeptide, or protein (from which it is derived), in particular a nucleic acid sequence or an amino acid sequence derived from another nucleic acid, peptide, polypeptide, or protein may be a functional sequence variant as described above of the starting nucleic acid, peptide, polypeptide, or protein (from which it is derived). For example, in a peptide/protein one or more amino acid residues may be substituted with other amino acid residues or one or more amino acid residue insertions or deletions may occur.

As used herein, the term “mutation” relates to a change in the nucleic acid sequence and/or in the amino acid sequence in comparison to a reference sequence, e.g., a corresponding genomic sequence. A mutation, e.g., in comparison to a genomic sequence, may be, for example, a (naturally occurring) somatic mutation, a spontaneous mutation, an induced mutation, e.g., induced by enzymes, chemicals, or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g., in a nucleic acid sequence or in an amino acid sequence. A mutation includes substitution, deletion, and insertion of one or more nucleotides or amino acids as well as inversion of several successive nucleotides or amino acids. Some types of coding sequence mutations include point mutations (differences in individual nucleotides or amino acids); silent mutations (differences in nucleotides that do not result in an amino acid changes); deletions (differences in which one or more nucleotides or amino acids are missing, up to and including a deletion of the entire coding sequence of a gene); frameshift mutations (differences in which deletion of a number of nucleotides indivisible by 3 results in an alteration of the amino acid sequence). A mutation that results in a difference in an amino acid may also be called an amino acid substitution mutation. Amino acid substitution mutations may be described by the amino acid change relative to wild type at a particular position in the amino acid sequence. To achieve a mutation in an amino acid sequence, a mutation may be introduced into the nucleotide sequence encoding said amino acid sequence in order to express a (recombinant) mutated polypeptide. A mutation may be achieved, e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

The term “recombinant”, as used herein (e.g., a recombinant protein, a recombinant nucleic acid, a recombinant antibody, etc.), refers to any molecule (protein, nucleic acid, antibody, etc.) that is prepared, expressed, created, or isolated by recombinant means, and which is not naturally occurring. With reference to a nucleic acid or polypeptide, “recombinant” refers to one that has a sequence that is not naturally occurring or has a sequence that is made by an artificial combination of two or more otherwise separated segments of sequence, for example a CMV vector comprising a heterologous antigen. This artificial combination is often accomplished by chemical synthesis or, more commonly, by the artificial manipulation of isolated segments of nucleic acids, e.g., by genetic engineering techniques. A recombinant polypeptide may also refer to a polypeptide that has been made using recombinant nucleic acids, including recombinant nucleic acids transferred to a host organism that is not the natural source of the polypeptide (for example, nucleic acids encoding polypeptides that form a CMV vector comprising a heterologous antigen).

As used herein, the term “vector” refers to a carrier by which into which nucleic acid molecules of particular sequence can be incorporated and then introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Vectors can be viral vectors, such as CMV vectors. Viral vectors may be constructed from wild type or attenuated virus, including replication deficient virus.

As the term “operably linked” is used herein, a first nucleic acid sequence is operably linked with a second nucleic acid sequence when the first nucleic acid sequence is placed in such a way that it has an effect upon the second nucleic acid sequence. Operably linked DNA sequences may be contiguous, or they may operate at a distance.

As used herein, the term “promoter” may refer to any of a number of nucleic acid control sequences that directs transcription of a nucleic acid. Typically, a eukaryotic promoter includes necessary nucleic acid sequences near the start site of transcription, such as, in the case of a polymerase II type promoter, a TATA element or any other specific DNA sequence that is recognized by one or more transcription factors. Expression by a promoter may be further modulated by enhancer or repressor elements. Numerous examples of promoters are available and well known to those of ordinary skill in the art. A nucleic acid comprising a promoter operably linked to a nucleic acid sequence that codes for a particular polypeptide may be termed an expression vector. Promoters can be from CMV genes, including but not limited to UL82 and UL78.

As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included. Where distinct designations are intended, it will be clear from the context.

As used herein, the term “microRNA” refers to a major class of biomolecules involved in control of gene expression. For example, in human heart, liver, or brain, miRNAs play a role in tissue specification or cell lineage decisions. In addition, miRNAs influence a variety of processes, including early development, cell proliferation and cell death, and apoptosis and fat metabolism. The large number of miRNA genes, the diverse expression patterns, and the abundance of potential miRNA targets suggest that miRNAs may be a significant source of genetic diversity. A mature miRNA is typically an 8-25 nucleotide non-coding RNA that regulates expression of an mRNA including sequences complementary to the miRNA. These small RNA molecules are known to control gene expression by regulating the stability and/or translation of mRNAs. For example, miRNAs bind to the 3′ UTR of target mRNAs and suppress translation. MiRNAs may also bind to target mRNAs and mediate gene silencing through the RNAi pathway. MiRNAs may also regulate gene expression by causing chromatin condensation.

A miRNA silences translation of one or more specific mRNA molecules by binding to a miRNA recognition element (MRE,) which is defined as any sequence that directly base pairs with and interacts with the miRNA somewhere on the mRNA transcript. Often, the MRE is present in the 3′ untranslated region (UTR) of the mRNA, but it may also be present in the coding sequence or in the 5′ UTR. MREs are not necessarily perfect complements to miRNAs, usually having only a few bases of complementarity to the miRNA and often containing one or more mismatches within those bases of complementarity. The MRE may be any sequence capable of being bound by a miRNA sufficiently that the translation of a gene to which the MRE is operably linked (such as a CMV gene that is essential or augmenting for growth in vivo) is repressed by a miRNA silencing mechanism such as the RISC.

The term “vaccine” as used herein is typically understood to be a prophylactic or therapeutic material providing at least one antigen or immunogen. The antigen or immunogen may be derived from any material that is suitable for vaccination. For example, the antigen or immunogen may be derived from a pathogen, such as from bacteria or virus particles, etc., or from a tumor or cancerous tissue. The antigen or immunogen stimulates the body's adaptive immune system to provide an adaptive immune response. In particular, an “antigen” or an “immunogen” refers typically to a substance which may be recognized by the immune system (e.g., the adaptive immune system), and which is capable of triggering an antigen-specific immune response, e.g., by formation of antibodies and/or antigen-specific T cells as part of an adaptive immune response. Typically, an antigen may be or may comprise a peptide or protein which may be presented by the MHC to T-cells. Vaccines can be used prophylactically or therapeutically. Thus, vaccines can be used reduce the likelihood of developing a disease (such as a tumor or pathological infection) or to reduce the severity of symptoms of a disease or condition, limit the progression of the disease or condition (such as a tumor or a pathological infection), or limit the recurrence of a disease or condition (such as a tumor). In particular embodiments, a vaccine comprises a replication-deficient CMV expressing a heterologous antigen. In particular embodiments, a vaccine comprises a replication-deficient CMV expressing a fusion protein comprising Mtb antigens. As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) the protein is able to evoke an immune response of the humoral and/or cellular type directed against that protein.

As used herein, “heterologous” or “exogenous” nucleic acid molecule, construct, or sequence refers to a nucleic acid molecule or portion of a nucleic acid molecule that is not native to a second nucleic acid molecule or to a host cell, depending on the context, but may be homologous to a nucleic acid molecule or portion of the second nucleic acid molecule or host cell. The source of the heterologous or exogenous nucleic acid molecule, construct, or sequence may be from a different genus or species, or may be synthetic. In certain embodiments, a heterologous or exogenous nucleic acid molecule is added (i.e., not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self replicating vector), and may be present in multiple copies. In addition, the term “heterologous” includes a non-native enzyme, protein, or other activity encoded by an exogenous nucleic acid molecule introduced into the second nucleic acid molecule or host cell, even if the second nucleic acid molecule or host cell encodes a homologous protein or activity.

As used herein, the term “heterologous antigen” refers to any protein or fragment thereof that is not derived from a vector to which it has been inserted. For example, in some embodiments, a “heterologous antigen” is any protein or fragment thereof that is not derived from CMV. Heterologous antigens may be pathogen-specific antigens, tumor virus antigens, tumor antigens, host self-antigens, or any other antigen.

As used herein, “antigen-specific T cell” refers to a CD8+ or CD4+ lymphocyte that recognizes a particular antigen. Generally, antigen-specific T cells specifically bind to a particular antigen presented by MHC molecules, but not other antigens presented by the same MHC.

As used herein, “immunogenic peptide” refers to peptide that comprises an allele-specific motif or other sequence, such as an N-terminal repeat, such that the peptide will bind an MHC molecule and induce a cytotoxic T lymphocyte (“CTL”) response, or a B cell response (for example, antibody production) against the antigen from which the immunogenic peptide is derived. In some embodiments, immunogenic peptides are identified using sequence motifs or other methods, such as neural net or polynomial determinations known in the art. Typically, algorithms are used to determine the “binding threshold” of peptides to select those with scores that give them a high probability of binding at a certain affinity and will be immunogenic. The algorithms are based either on the effects on MHC binding of a particular amino acid at a particular position, the effects on antibody binding of a particular amino acid at a particular position, or the effects on binding of a particular substitution in a motif-containing peptide. Within the context of an immunogenic peptide, a “conserved residue” is one which appears in a significantly higher frequency than would be expected by random distribution at a particular position in a peptide. In some embodiments, a conserved residue is one where the MHC structure may provide a contact point with the immunogenic peptide.

As used herein, the term “administration” means to provide or give a subject an agent, such as a composition comprising an effective amount of an antigen or pharmaceutical composition comprising an exogenous antigen by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

As used herein, a “pharmaceutically acceptable carrier” of use is conventional. Remington's Pharmaceutical Sciences, by E. W. Martin, Mack Publishing Co., Easton, PA, 19th Edition, 1995, describes compositions and formulations suitable for pharmaceutical delivery of the compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (such as powder, pill, tablet, or capsule forms), conventional non-toxic solid carriers may include, for example, pharmaceutical grades of mannitol, lactose, starch, or magnesium stearate. In addition to biologically neutral carriers, pharmaceutical compositions to be administered may contain minor amounts of non-toxic auxiliary substances, such as wetting or emulsifying agents, preservatives, and pH buffering agents and the like, for example sodium acetate or sorbitan monolaurate.

Doses are often expressed in relation to bodyweight. Thus, a dose which is expressed as [g, mg, or other unit]/kg (or g, mg, etc.) usually refers to [g, mg, or other unit] “per kg (or g, mg, etc.) bodyweight”, even if the term “bodyweight” is not explicitly mentioned.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, “tuberculosis” means a disease that is generally caused by Mycobacterium tuberculosis that usually infects the lungs. However, other “atypical” mycobacteria such as M. kansasii may produce a similar clinical and pathologic appearance of disease. Transmission of M. tuberculosis occurs by the airborne route in confined areas with poor ventilation. In more than 90% of cases, following infection with M. tuberculosis, the immune system prevents development of disease from M. tuberculosis, often called, active tuberculosis. However, not all of the M. tuberculosis is killed and, thus tiny, hard capsules are formed. “Primary tuberculosis” is seen as disease that develops following an initial infection, usually in children. The initial focus of infection is a small subpleural granuloma accompanied by granulomatous hilar lymph node infection. Together, these make up the Ghon complex. In nearly all cases, these granulomas resolve and there is no further spread of the infection. “Secondary tuberculosis” is seen mostly in adults as a reactivation of previous infection (or reinfection), particularly when health status declines. The granulomatous inflammation is much more florid and widespread. Typically, the upper lung lobes are most affected, and cavitation can occur. Dissemination of tuberculosis outside of the lungs can lead to the appearance of a number of uncommon findings with characteristic patterns that include skeletal tuberculosis, genital tract tuberculosis, urinary tract tuberculosis, central nervous system (CNS) tuberculosis, gastrointestinal tuberculosis, adrenal tuberculosis, scrofula, and cardiac tuberculosis. “Latent” tuberculosis is an Mtb infection in an individual that can be detected by a diagnostic assay, such as, but not limited to a tuberculin skin test (TST) wherein the infection does not produce symptoms in that individual. “Active” tuberculosis is a symptomatic Mtb infection in a subject. Microscopically, the inflammation produced with TB infection is granulomatous, with epithelioid macrophages and Langhans giant cells along with lymphocytes, plasma cells, maybe a few polymorphonuclear cells, fibroblasts with collagen, and characteristic caseous necrosis in the center. The inflammatory response is mediated by a type IV hypersensitivity reaction, and skin testing is based on this reaction. In some examples, tuberculosis can be diagnosed by a skin test, an acid fast stain, an auramine stain, or a combination thereof. The most common specimen screened is sputum, but the histologic stains can also be performed on tissues or other body fluids. “Pulmonary” tuberculosis refers to any bacteriologically confirmed or clinically diagnosed case of tuberculosis involving the lungs, including the lung parenchyma and/or the tracheobronchial tree. “Extrapulmonary” tuberculosis refers to any bacteriologically confirmed or clinically diagnosed case of tuberculosis involving organs other than the lungs, including, but not limited to, the pleura, lymph nodes, abdomen, genitourinary tract, skin, joints, bones, and/or meninges.

As used herein, “recurrent” tuberculosis, refers to the reactivation of an endogenous, primary M. tuberculosis infection or to recent exogenous re-infection with M. tuberculosis. Recurrent tuberculosis also refers to “postprimary tuberculosis” which may occur many years after a primary infection.

As used herein, “adjunct” refers to a treatment used together with the primary treatment wherein the purpose of the adjunct treatment is to assist the primary treatment.

### II. Tuberculosis Antigens

Disclosed herein are fusion proteins comprising Mycobacterium tuberculosis (Mtb) antigens and nucleic acids encoding the same.

In some embodiments, the present disclosure provides a fusion protein comprising one or more of Mtb Ag85A, ESAT-6, Rv3407, Rv2626c, RpfA, RpfD, Ra12, TbH9, and Ra35, or portions or fragments thereof.

Ag85A is a Mtb acetyltransferase enzyme that forms a complex with Ag85B and Ag85C and is involved in the synthesis of components of the mycobacterial cell envelope (see, e.g., Elamin, A A et al., The Mycobacterium tuberculosis Ag85A is a novel diacylglycerol acyltransferase involved in lipid body formation. Molecular Microbiology 81, 1577-1592 (2011)). As used herein, “Ag85A” may refer to the enzyme or an amino acid sequence encoding an Ag85A protein or peptide, or portions thereof, depending on the context. In some embodiments, Ag85A refers to an amino acid sequence according to UniProtKB-P9WQP3 (A85A_MYCTU) (SEQ ID NO:1). In some embodiments, Ag85A refers to a fragment of the amino acid sequence according to SEQ ID NO:1. In some embodiments, Ag85A refers to the amino acid sequence according to SEQ ID NO:11. In some embodiments, Ag85A refers to the amino acid sequence according to SEQ ID NO:12. In some embodiments, Ag85A refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:1, or a fragment thereof. In some embodiments, Ag85A refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:11. In some embodiments, Ag85A refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:12.

ESAT-6 is a secreted Mtb protein associated with pathogenic virulence and modulation of host immune responses (Sreejit, G et al. The ESAT-6 Protein of Mycobacterium tuberculosis Interacts with Beta-2-Microglobulin (02M) Affecting Antigen Presentation Function of Macrophage. PLoS Pathog 10, e1004446 (2014)). As used herein, “ESAT-6” may refer to the protein or peptide or an amino acid sequence encoding an ESAT-6 protein or peptide, or portions thereof, depending on the context. In some embodiments, ESAT-6 refers to the amino acid sequence according to UniProtKB-P9WNK7 (ESXA_MYCTU) (SEQ ID NO:2). In some embodiments, ESAT-6 refers to a fragment of the amino acid sequence according to SEQ ID NO:2. In some embodiments, ESAT-6 refers to the amino acid sequence according to SEQ ID NO:13. In some embodiments, ESAT-6 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NOs:2, or a fragment thereof. In some embodiments, ESAT-6 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:13.

Rv3407 is a Mtb antigen (Mollenkopf, H J et al. Application of Mycobacterial Proteomics to Vaccine Design: Improved Protection by Mycobacterium bovis BCG Prime-Rv3407 DNA Boost Vaccination against Tuberculosis. Infection and Immunity 72, 6471-6479 (2004)). As used herein, “Rv3407” may refer to the antigenic protein or peptide or an amino acid sequence encoding an Rv3407 protein or peptide, or portions thereof, depending on the context. In some embodiments, Rv3407 refers to the amino acid sequence according to UniProtKB-P9WF23 (VPB47_MYCTU) (SEQ ID NO:3). In some embodiments, Rv3407 refers to a fragment of the amino acid sequence according to SEQ ID NO:3. In some embodiments, Rv3407 refers to the amino acid sequence according to SEQ ID NO: 14. In some embodiments, Rv3407 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NOs:3, or a fragment thereof. In some embodiments, Rv3407 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:14.

Rv2626c has been identified as a Mtb latency antigen (Amiano, N O et al. IFN-γ and IgG responses to Mycobacterium tuberculosis latency antigen Rv2626c differentiate remote from recent tuberculosis infection. Sci Rep 10, 7472 (2020)). As used herein, “Rv2626c” may refer to the antigenic protein or peptide or an amino acid sequence encoding an Rv2626c protein or peptide, or portions thereof, depending on the context. In some embodiments, Rv2626c refers to the amino acid sequence according to UniProtKB-P9WJA3 (HRP1_MYCTU) (SEQ ID NO:4). In some embodiments, Rv2626c refers to a fragment of the amino acid sequence according to SEQ ID NO:4. In some embodiments, Rv2626c refers to the amino acid sequence according to SEQ ID NO:15. In some embodiments, Rv2626c refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NOs:4, or a fragment thereof. In some embodiments, Rv2626c refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:15.

RpfA and RpfD belong to a family of Mtb proteins involved in virulence and resuscitation from dormancy but generally not necessary for in vitro growth (Kana, B D et al. The resuscitation-promoting factors of Mycobacterium tuberculosis are required for virulence and resuscitation from dormancy but are collectively dispensable for growth in vitro. Mol Microbiol. 67, 672-684 (2008)). As used herein, “RpfA” may refer to the protein or peptide or an amino acid sequence encoding an RpfA protein or peptide, or portions thereof, depending on the context. RpfA has variable expression in Mtb strains. RpfA in Mtb strains may be full-length or include only the C-terminus, only the N-terminus, and/or lack a central portion of the protein. In some embodiments, RpfA refers to the amino acid sequence according to UniProtKB-P9WG31 (RPFA_MYCTU) (SEQ ID NO:5). In some embodiments, RpfA refers to a fragment of the amino acid sequence according to SEQ ID NO:5. In some embodiments, RpfA refers to the amino acid sequence according to SEQ ID NO:16. In some embodiments, RpfA refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NOs:5, or a fragment thereof. In some embodiments, RpfA refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:16.

As used herein, “RpfD” may refer to the protein or peptide or an amino acid sequence encoding an RpfD protein or peptide, or portions thereof, depending on the context. In some embodiments, RpfD refers to the amino acid sequence according to UniProtKB-P9WG27 (RPFD_MYCTU) (SEQ ID NO:6). In some embodiments, RpfD refers to a fragment of the amino acid sequence according to SEQ ID NO:6. In some embodiments, RpfD refers to the amino acid sequence according to SEQ ID NO:17. In some embodiments, RpfD refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NOs:6, or a fragment thereof. In some embodiments, RpfD refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:17.

Ra12 refers to a C-terminal portion of Mtb32A, typically comprising the last approximately 132 amino acids of Mtb32A (International Application Publication No. WO2006/117240A2, which is incorporated herein by reference for teachings related to Ra12 and Ra35 antigens and fusions comprising the same). Mtb32A is a Mtb serine protease (Skeiky, Y A W et al. Cloning, Expression, and Immunological Evaluation of Two Putative Secreted Serine Protease Antigens of Mycobacterium tuberculosis. Infection and Immunity 67, 3998-4007 (1999)). An example is the Mtb32A sequence corresponding to UniProtKB-007175 (007175_MYCTU) (SEQ ID NO:7). As used herein, “Ra12” may refer to the protein or peptide or an amino acid sequence encoding a Ra12 protein or peptide, or portions thereof, depending on the context. In some embodiments, Ra12 refers to the amino acid sequence according to SEQ ID NO:23. In some embodiments, Ra12 refers to a fragment of the amino acid sequence according to SEQ ID NO:23. In some embodiments, Ra12 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:23.

Ra35 refers to an N-terminal portion of Mtb32A (International Application Publication No. WO2006/117240A2). As used herein, “Ra35” may refer to the protein or peptide or an amino acid sequence encoding a Ra35 protein or peptide, or portions thereof, depending on the context. In some embodiments, Ra35 refers to the amino acid sequence according to SEQ ID NO:25. In some embodiments, Ra35 refers to a fragment of the amino acid sequence according to SEQ ID NO:25. In some embodiments, Ra35 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:25.

In some embodiments, Ra35 refers to an amino acid sequence according to SEQ ID NO:26. In some embodiments, Ra35 refers to a fragment of the amino acid sequence according to SEQ ID NO:26. In some embodiments, Ra35 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:26.

TbH9 (also known as Mtb39a or PPE18) is a member of the mycobacterial proline-proline-glutamic acid (PPE) family of proteins and appears to be involved in intracellular survival of Mtb (Bhat, K H et al. Role of PPE18 Protein in Intracellular Survival and Pathogenicity of Mycobacterium tuberculosis in Mice. PLoS ONE 7, e52601 (2012)). TbH9 (Mtb39a) is highly homologous to Mtb39b and Mtb39c, which, together comprise the Mtb39 gene family. As used herein, “TbH9” may refer to the protein or peptide or an amino acid sequence encoding an TbH9 protein or peptide, or portions thereof, depending on the context. In some embodiments, TbH9 refers to the amino acid sequence according to UniProtKB-L7N675 (PPE18_MYCTU) (SEQ ID NO:8). In some embodiments, TbH9 refers to a fragment of the amino acid sequence according to SEQ ID NO:8. In some embodiments, TbH9 refers to the amino acid sequence according to SEQ ID NO:24. In some embodiments, TbH9 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NOs:8, or a fragment thereof. In some embodiments, TbH9 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:24.

Mtb72f is a fusion protein comprising M. tuberculosis proteins Mtb32a and TbH9. Mtb72f was constructed by fusing TbH9 with C- and N-terminal portions of Mtb32a as follows: Mtb32 C-terminal end-Mtb39- Mtb32 N-terminal end. An open reading frame (ORF) encoding an approximately 14-kDa C-terminal fragment of Mtb32a was sequentially linked to the full length ORF of TbH9 followed by an approximately 20-kDa N-terminal portion of Mtb32a. As used herein, “Mtb72f” may refer to the antigenic fusion protein or peptide or an amino acid sequence encoding an Mtb72f protein or peptide, or portions thereof, depending on the context. In some embodiments, Mtb72f refers to the amino acid sequence according to SEQ ID NO:18. In some embodiments, Mtb72f refers to a fragment of the amino acid sequence according to SEQ ID NO:18. In some embodiments, Mtb72f refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:18, or a fragment thereof.

Mtb72f may also refer to an Mtb72f wherein the methionine at amino acid position 1 has been removed according to SEQ ID NO: 19. In some embodiments, Mtb72f refers to a fragment of the amino acid sequence according to SEQ ID NO:19. In some embodiments, Mtb72f refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:19, or a fragment thereof.

M72 is a variant of Mtb72f that includes a two-residue histidine tag at the N-terminus and a serine to alanine substitution at amino acid position 710. The C-terminal portion of Mtb32a and the full length ORF of TbH9 are otherwise the same sequence as in Mtb72f (SEQ ID NO:18). The N-terminal portion of Mtb32a contains the S710A substitution. As used herein, “M72” may refer to the antigenic fusion protein or peptide or an amino acid sequence encoding an M72 protein or peptide, or portions thereof, depending on the context. In some embodiments, M72 refers to the amino acid sequence according to SEQ ID NO:21. In some embodiments, M72 refers to a fragment of the amino acid sequence according to SEQ ID NO:21. In some embodiments, M72 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:21, or a fragment thereof.

“M72-fusion-2” is a variant of M72 with a 3 amino acid deletion at the N terminus, wherein the methionine at amino acid position 1 and the two-residue histidine tag have both been removed. In some embodiments, M72-fusion-2 refers to the amino acid sequence according to SEQ ID NO:22. In some embodiments, M72-fusion-2 refers to a fragment of the amino acid sequence according to SEQ ID NO:22. In some embodiments, M72-fusion-2 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:22, or a fragment thereof.

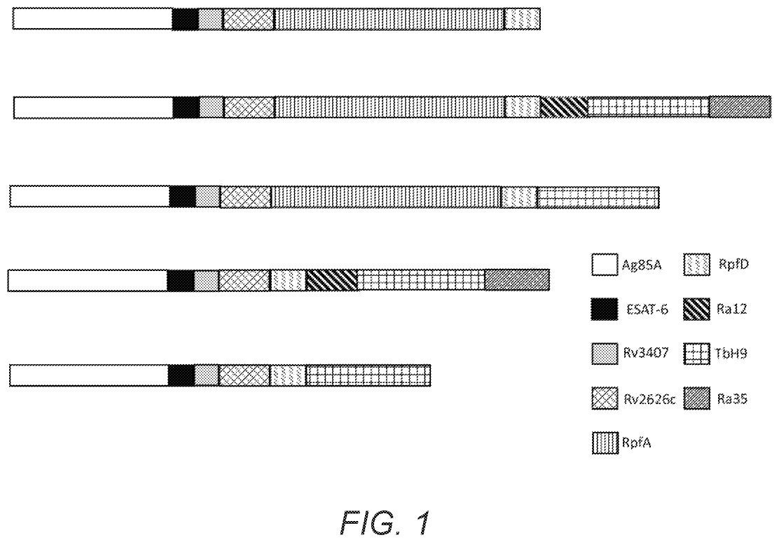

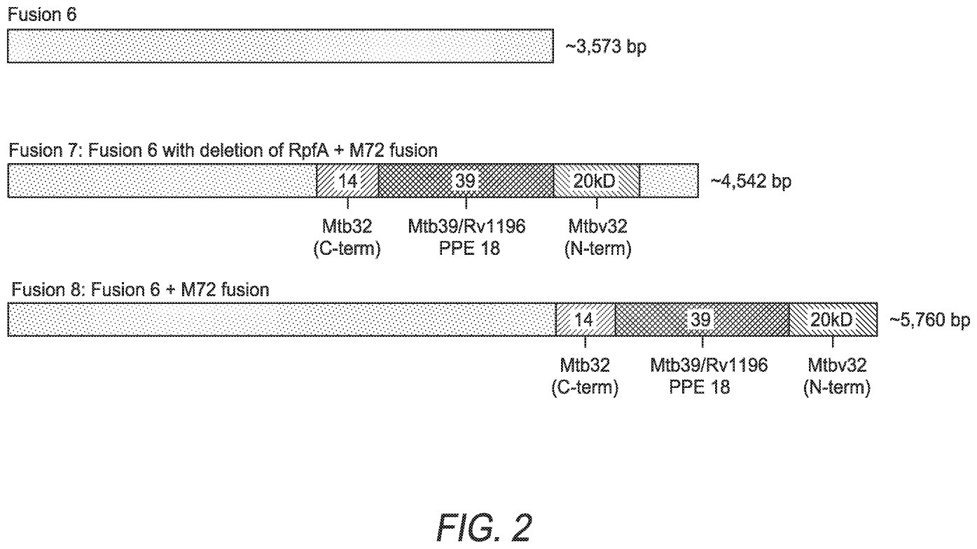

FIGS. 1 and 2 show non-limiting examples of fusion proteins described herein.

In some embodiments, the present disclosure provides a fusion protein comprising, consisting, or consisting essentially of Ag85A, ESAT-6, Rv3407, Rv2626c, Ra12, TbH9, Ra35, and RpfD, or fragments thereof. In some embodiments, the present disclosure provides an Ag85A-ESAT-6-Rv3407-Rv2626c-Ra12-TbH9-Ra35-RpfD fusion protein. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:42. In some embodiments, the fusion protein comprises the amino acid sequence according to SEQ ID NO:42.

In some embodiments, the present disclosure provides a fusion protein comprising, consisting, or consisting essentially of Ag85A, ESAT-6, Rv3407, Rv2626c, RpfA, and RpfD or fragments thereof. In some embodiments, the present disclosure provides an Ag85A-ESAT-6-Rv3407-Rv2626c-RpfA-RpfD fusion protein. In some embodiments, the fusion protein comprises an amino acid sequence having 90%, 91%, 92%, 93%, 94%, 95%, 96%, 97%, 98%, 99%, or 100% identity to the amino acid sequence according to any one of SEQ ID NOs:9-10. In some embodiments, the fusion protein comprises the amino acid sequence according to SEQ ID NO:9. In some embodiments, the fusion protein comprises the amino acid sequence according to SEQ ID NO:10.

In some embodiments, the present disclosure provides a fusion protein comprising, consisting, or consisting essentially of Ag85A, ESAT-6, Rv3407, Rv2626c, RpfA, RpfD, Ra12, TbH9, and Ra35, or fragments thereof. Unless otherwise specified, the individual Mtb antigens can be present in the fusion protein in any order. Additionally, they may be connected in a C-terminus to N-terminus to C-terminus manner, with or without linkers as described herein. In some embodiments, the present disclosure provides a Ag85A-ESAT-6-Rv3407-Rv2626c-RpfA-RpfD-Ra12-TbH9-Ra35 fusion protein. In some embodiments, the fusion protein comprises (i) an amino acid sequence having 90%, 91%, 92%, 93%, 94%, 95%, 96%, 97%, 98%, 99%, or 100% identity to the amino acid sequence according to any one of SEQ ID NOs:9-10; and (ii) an amino acid sequence having 90%, 91%, 92%, 93%, 94%, 95%, 96%, 97%, 98%, 99%, or 100% identity to the amino acid sequence according to any one of SEQ ID NOs:18-22. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:27. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:28. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:29. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:30. In some embodiments, the fusion protein comprises the amino acid sequence according to SEQ ID NO:27. In some embodiments, the fusion protein comprises the amino acid sequence according to SEQ ID NO:28. In some embodiments, the fusion protein comprises the amino acid sequence according to SEQ ID NO:29. In some embodiments, the fusion protein comprises the amino acid sequence according to SEQ ID NO:30.

In some embodiments, the present disclosure provides a fusion protein comprising, consisting, or consisting essentially of Ag85A, ESAT-6, Rv3407, Rv2626c, RpfA, RpfD, and TbH9, or fragments thereof. In some embodiments, the present disclosure provides a Ag85A-ESAT-6-Rv3407-Rv2626c-RpfA-RpfD-TbH9 fusion protein. In some embodiments, the fusion protein comprises (i) an amino acid sequence having 90%, 91%, 92%, 93%, 94%, 95%, 96%, 97%, 98%, 99%, or 100% identity to the amino acid sequence according to any one of SEQ ID NOs:9-10; and (ii) an amino acid sequence having 90%, 91%, 92%, 93%, 94%, 95%, 96%, 97%, 98%, 99%, or 100% identity to the amino acid sequence according to SEQ ID NO:24. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:31; In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:32. In some embodiments, the fusion protein comprises the amino acid sequence according to SEQ ID NO:31. In some embodiments, the fusion protein comprises the amino acid sequence according to SEQ ID NO:32.

In some embodiments, the present disclosure provides a fusion protein comprising, consisting, or consisting essentially of Ag85A, ESAT-6, Rv3407, Rv2626c, RpfD, Ra12, TbH9, and Ra35, or fragments thereof. In some embodiments, the present disclosure provides a Ag85A-ESAT-6-Rv3407-Rv2626c-RpfD-Ra12-TbH9-Ra35 fusion protein. In some embodiments, the fusion protein comprises (i) an amino acid sequence having 90%, 91%, 92%, 93%, 94%, 95%, 96%, 97%, 98%, 99%, or 100% identity to the amino acid sequence according to any one of SEQ ID NOs: 1 and 11-12; (ii) an amino acid sequence having 90%, 91%, 92%, 93%, 94%, 95%, 96%, 97%, 98%, 99%, or 100% identity to the amino acid sequence according to SEQ ID NO:2 or 13; (iii) an amino acid sequence having 90%, 91%, 92%, 93%, 94%, 95%, 96%, 97%, 98%, 99%, or 100% identity to the amino acid sequence according to SEQ ID NO:3 or 14; (iv) an amino acid sequence having 90%, 91%, 92%, 93%, 94%, 95%, 96%, 97%, 98%, 99%, or 100% identity to the amino acid sequence according to SEQ ID NO:4 or 15; (v) an amino acid sequence having 90%, 91%, 92%, 93%, 94%, 95%, 96%, 97%, 98%, 99%, or 100% identity to the amino acid sequence according to SEQ ID NO:6 or 17; (vi) an amino acid sequence having 90%, 91%, 92%, 93%, 94%, 95%, 96%, 97%, 98%, 99%, or 100% identity to the amino acid sequence according to SEQ ID NO:23; (vii) an amino acid sequence having 90%, 91%, 92%, 93%, 94%, 95%, 96%, 97%, 98%, 99%, or 100% identity to the amino acid sequence according to SEQ ID NO:8 or 24; and (viii) an amino acid sequence having 90%, 91%, 92%, 93%, 94%, 95%, 96%, 97%, 98%, 99%, or 100% identity to the amino acid sequence according to any one of SEQ ID NOs:25-26. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:33. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:34. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:35. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:36. In some embodiments, the fusion protein comprises the amino acid sequence according to SEQ ID NO:33. In some embodiments, the fusion protein comprises the amino acid sequence according to SEQ ID NO:34. In some embodiments, the fusion protein comprises the amino acid sequence according to SEQ ID NO:35. In some embodiments, the fusion protein comprises the amino acid sequence according to SEQ ID NO:36.

In some embodiments, the present disclosure provides a fusion protein comprising, consisting, or consisting essentially of Ag85A, ESAT-6, Rv3407, Rv2626c, RpfD, and TbH9, or fragments thereof. In some embodiments, the present disclosure provides a Ag85A-ESAT-6-Rv3407-Rv2626c-RpfD-TbH9 fusion protein. In some embodiments, the fusion protein comprises (i) an amino acid sequence having 90%, 91%, 92%, 93%, 94%, 95%, 96%, 97%, 98%, 99%, or 100% identity to the amino acid sequence according to any one of SEQ ID NOs:1 and 11-12; (ii) an amino acid sequence having 90%, 91%, 92%, 93%, 94%, 95%, 96%, 97%, 98%, 99%, or 100% identity to the amino acid sequence according to SEQ ID NO:2 or 13; (iii) an amino acid sequence having 90%, 91%, 92%, 93%, 94%, 95%, 96%, 97%, 98%, 99%, or 100% identity to the amino acid sequence according to SEQ ID NO:3 or 14; (iv) an amino acid sequence having 90%, 91%, 92%, 93%, 94%, 95%, 96%, 97%, 98%, 99%, or 100% identity to the amino acid sequence according to SEQ ID NO:4 or 15; (v) an amino acid sequence having 90%, 91%, 92%, 93%, 94%, 95%, 96%, 97%, 98%, 99%, or 100% identity to the amino acid sequence according to SEQ ID NO:6 or 17; and (vi) an amino acid sequence having 90%, 91%, 92%, 93%, 94%, 95%, 96%, 97%, 98%, 99%, or 100% identity to the amino acid sequence according to SEQ ID NO:8 or 24. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:37. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:38. In some embodiments, the fusion protein comprises the amino acid sequence according to SEQ ID NO:37. In some embodiments, the fusion protein comprises the amino acid sequence according to SEQ ID NO:38.In some embodiments, the fusion protein comprises an amino acid sequence having at least 90%, at least 91%, at least 92%, at least 93%, at least 94%, at least 95%, at least 96%, at least 97%, at least 98%, at least 99%, or 100% identity to the amino acid sequence according to any one of SEQ ID NOs:42 and 1-38.

In some embodiments, the fusion protein comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:42. In some embodiments, the fusion protein consists of the amino acid sequence according to SEQ ID NO:42. In some embodiments, the fusion protein consists essentially of the amino acid sequence according to SEQ ID NO:42.

In some embodiments, the fusion protein comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:27. In some embodiments, the fusion protein comprises the amino acid sequence according to SEQ ID NO:27. In some embodiments, the fusion protein consists of the amino acid sequence according to SEQ ID NO:27. In some embodiments, the fusion protein consists essentially of the amino acid sequence according to SEQ ID NO:27.

In some embodiments, the fusion protein comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:28. In some embodiments, the fusion protein comprises the amino acid sequence according to SEQ ID NO:28. In some embodiments, the fusion protein consists of the amino acid sequence according to SEQ ID NO:28. In some embodiments, the fusion protein consists essentially of the amino acid sequence according to SEQ ID NO:28.

In some embodiments, the fusion protein comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:29. In some embodiments, the fusion protein comprises the amino acid sequence according to SEQ ID NO:29. In some embodiments, the fusion protein consists of the amino acid sequence according to SEQ ID NO:29. In some embodiments, the fusion protein consists essentially of the amino acid sequence according to SEQ ID NO:29.

In some embodiments, the fusion protein comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:30. In some embodiments, the fusion protein comprises the amino acid sequence according to SEQ ID NO:30. In some embodiments, the fusion protein consists of the amino acid sequence according to SEQ ID NO:30. In some embodiments, the fusion protein consists essentially of the amino acid sequence according to SEQ ID NO:30.

In some embodiments, the fusion protein comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:31. In some embodiments, the fusion protein comprises the amino acid sequence according to SEQ ID NO:31. In some embodiments, the fusion protein consists of the amino acid sequence according to SEQ ID NO:31. In some embodiments, the fusion protein consists essentially of the amino acid sequence according to SEQ ID NO:31.

In some embodiments, the fusion protein comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:32. In some embodiments, the fusion protein comprises the amino acid sequence according to SEQ ID NO:32. In some embodiments, the fusion protein consists of the amino acid sequence according to SEQ ID NO:32. In some embodiments, the fusion protein consists essentially of the amino acid sequence according to SEQ ID NO:32.

In some embodiments, the fusion protein comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:33. In some embodiments, the fusion protein comprises the amino acid sequence according to SEQ ID NO:33. In some embodiments, the fusion protein consists of the amino acid sequence according to SEQ ID NO:33. In some embodiments, the fusion protein consists essentially of the amino acid sequence according to SEQ ID NO:33.

In some embodiments, the fusion protein comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:34. In some embodiments, the fusion protein comprises the amino acid sequence according to SEQ ID NO:34. In some embodiments, the fusion protein consists of the amino acid sequence according to SEQ ID NO:34. In some embodiments, the fusion protein consists essentially of the amino acid sequence according to SEQ ID NO:34.

In some embodiments, the fusion protein comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:35. In some embodiments, the fusion protein comprises the amino acid sequence according to SEQ ID NO:35. In some embodiments, the fusion protein consists of the amino acid sequence according to SEQ ID NO:35. In some embodiments, the fusion protein consists essentially of the amino acid sequence according to SEQ ID NO:35.

In some embodiments, the fusion protein comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:36. In some embodiments, the fusion protein comprises the amino acid sequence according to SEQ ID NO:36. In some embodiments, the fusion protein consists of the amino acid sequence according to SEQ ID NO:36. In some embodiments, the fusion protein consists essentially of the amino acid sequence according to SEQ ID NO:36.

In some embodiments, the fusion protein comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:37. In some embodiments, the fusion protein comprises the amino acid sequence according to SEQ ID NO:37. In some embodiments, the fusion protein consists of the amino acid sequence according to SEQ ID NO:37. In some embodiments, the fusion protein consists essentially of the amino acid sequence according to SEQ ID NO:37.

In some embodiments, the fusion protein comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:38. In some embodiments, the fusion protein comprises the amino acid sequence according to SEQ ID NO:38. In some embodiments, the fusion protein consists of the amino acid sequence according to SEQ ID NO:38. In some embodiments, the fusion protein consists essentially of the amino acid sequence according to SEQ ID NO:38.

In any of the aforementioned embodiments, the fusion protein may further comprise a tag. In some embodiments, the fusion protein may further comprise a poly-His tag. In some embodiments, the poly-His tag comprises or consists of two to six His residues. In some embodiments, the poly-His tag is located at the N-terminus of the fusion protein or is inserted after an initial Met residue at the N-terminus. In any of the aforementioned embodiments, the fusion protein may further comprise a human influenza hemagglutinin (HA) tag comprising the amino acid sequence YPYDVPDYA (SEQ ID NO:40). In some embodiments, the HA tag is located at the C-terminus of the fusion protein. In some embodiments, the fusion protein may be conjugated to a tag or other imaging agent, such as biotin, fluorescent moieties, radioactive moieties, or other peptide tags.

Individual Mtb antigens may be linked together in a C-terminus to N-terminus or N- terminus to C-terminus manner without any linker. Alternately, a linker may be present between any two Mtb antigens within any of the fusion proteins disclosed herein. In some embodiments, the fusion protein may further comprise one or more linkers connecting one or more of Ag85A, ESAT-6, Rv3407, Rv2626c, RpfA, RpfD, Ra12, TbH9, and Ra35. For example, the linkers may comprise or consist of one or more amino acid residues included at the junction of two Mtb antigens. In some embodiments, the linker is encoded by a segment of DNA optionally containing one or more restrictions sites, wherein the segment of DNA is inserted between nucleic acid molecules encoding two Mtb antigens of any of the fusion proteins disclosed herein. In some embodiments, the restriction site comprises an EcoRI restriction site or an EcoRV restriction site. In some embodiments, the fusion protein comprises a linker between Ra12 and TbH9, resulting in an EcoRI restriction site. In some embodiments, the fusion protein comprises a linker between TbH9 and Ra35, resulting in an EcoRV restriction site.

In some aspects, the present disclosure provides a nucleic acid molecule encoding any of the fusion proteins disclosed herein.

In some embodiments, the fusion protein is encoded by a nucleic acid comprising a sequence having 90%, 91%, 92%, 93%, 94%, 95%, 96%, 97%, 98%, 99%, or 100% identity to the nucleic acid sequence according to SEQ ID NO:41. In some embodiments, the fusion protein consists of an amino acid sequence encoded by the nucleic acid sequence according to SEQ ID NO:41. In some embodiments, the fusion protein consists essentially of an amino acid sequence encoded by the nucleic acid sequence according to SEQ ID NO:41.

### III. Vectors

In some embodiments, the present disclosure provides vectors encoding a fusion protein as described above.

The vector may be any expression vector known in the art. For the antigens to be expressed, the protein coding sequence of the fusion protein should be “operably linked” to regulatory or nucleic acid control sequences that direct transcription and translation of the protein. A coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” may be any nucleic acid element, such as, but not limited to promoters, enhancers, IRES, introns, and other elements described herein that direct the expression of a nucleic acid sequence or coding sequence that is operably linked thereto. The term “promoter” refers to a group of transcriptional control modules that are clustered around the initiation site for RNA polymerase II and that when operationally linked to the protein coding sequences of the disclosure lead to the expression of the encoded protein. The expression of heterologous antigens and fusion proteins of the present disclosure may be under the control of a constitutive promoter or of an inducible promoter, which initiates transcription only when exposed to some particular external stimulus, such as, without limitation, antibiotics such as tetracycline, hormones such as ecdysone, or heavy metals. The promoter may also be specific to a particular cell-type, tissue, or organ. Many suitable promoters and enhancers are known in the art, and any such suitable promoter or enhancer may be used for expression of the transgenes of the disclosure. For example, suitable promoters and/or enhancers may be selected from the Eukaryotic Promoter Database (EPDB).

In some embodiments, the vector encoding the fusion protein is a plasmid, cosmid, phage, bacterial vector, or viral vector. In some embodiments, the vector is a viral vector, such a poxvirus, adenovirus, rubella, sendai virus, rhabdovirus, alphavirus, herpesvirus, lentivirus, retrovirus, or adeno-associated virus. In some embodiments, the vector encoding the fusion protein is a bacterial artificial chromosome (BAC). In some embodiments, the vector encoding the fusion protein is a CMV vector, e.g., a RhCMV or HCMV vector. In some embodiments, the vector encoding the fusion protein is a recombinant HCMV vector comprising a TR3 backbone.

In some embodiments, the recombinant CMV vector is or is derived from HCMV TR3. As referred to herein, “HCMV TR3” or “TR3” refers to a HCMV-TR3 vector backbone derived from the clinical isolate HCMV TR, as described in Caposio, P et al. (Characterization of a live attenuated HCMV-based vaccine platform. Scientific Reports 9, 19236 (2019)).

As described herein, recombinant CMV vectors may be characterized by the presence or absence of one or more CMV genes. CMV vectors may also be characterized by the presence or absence of one or more proteins encoded by one or more CMV genes. A protein encoded by a CMV gene may be absent due to the presence of a mutation in the nucleic acid sequence encoding the CMV gene. In some embodiments, the vector can include an ortholog or homolog of a CMV gene. Examples of CMV genes include, but are not limited to, UL82, UL128, UL130, UL146, UL147, UL 18, and UL78.

The human cytomegalovirus UL82 gene encodes pp71, a protein that is localized in the tegument domain of the virus particle. For example, the UL82 gene of the CMV TR strain is 118811 to 120490 for GenBank Accession No. KF021605.1.

Pp71 may perform one or more functions, including inhibition of Daxx repression of viral gene transcription, negative regulation of STING, and evasion of cell antiviral responses (Kalejta R F, et al. Expanding the Known Functional Repertoire of the Human Cytomegalovirus pp71 Protein. Front Cell Infect Microbiol. 2020 Mar. 12; 10:95). Deletion of UL82 or disruption of UL82 by insertion of a foreign gene at the UL82 locus results in the absence of pp71 protein and consequently reduces replication in fibroblasts, endothelial cells, epithelial cells, and astrocytes (Caposio P et al., Characterization of a live-attenuated HCMV-based vaccine platform. Sci Rep. 2019 Dec. 17; 9(1):19236). The effects of UL82 deletion or disruption are reversible by cell kinase inhibitors. The rhesus cytomegalovirus (RhCMV) gene RhCMV 110 is homologous to human CMV UL82 (Hansen S G, et al. Complete sequence and genomic analysis of rhesus cytomegalovirus. J Virol. 2003 June; 77(12):6620-36).

The human cytomegalovirus genes UL128 and UL130 encode structural components of the viral envelope (Patrone, M et al. Human cytomegalovirus UL130 protein promotes endothelial cell infection through a producer cell modification of the virion. J Virol. 79(13):8361-73 (2005); Ryckman, B J et al. Characterization of the human cytomegalovirus gH/gL/UL128-131 complex that mediates entry into epithelial and endothelial cells. J Virol. 82(1):60-70 (2008); Wang, D et al. Human cytomegalovirus virion protein complex required for epithelial and endothelial cell tropism. Proc Natl Acad Sci USA. 102(50):18153-8 (2005)). For example, the UL128 gene of the CMV TR strain is 176206 to 176964 for GenBank Accession No. KF021605.1 and the UL130 gene of the CMV TR strain is 177004 to 177648 for GenBank Accession No. KF021605.1.

The human cytomegalovirus genes UL146 and UL147 encode the CXC chemokines vCXC-1 and vCXC-2, respectively (Penfold, M E et al. Cytomegalovirus encodes a potent alpha chemokine. Proc Natl Acad Sci USA. 96(17):9839-44 (1999)). For example, the UL146 gene of the CMV TR strain is 180954 to 181307 for GenBank Accession No. KF021605.1 and the UL147 gene of the CMV TR strain is 180410 to 180889 for GenBank Accession No. KF021605.1.

The human cytomegalovirus UL18 gene encodes a type-I membrane glycoprotein that associates with 02-microglobulin and can bind endogenous peptides (Park, B et al. Human cytomegalovirus inhibits tapasin-dependent peptide loading and optimization of the MHC class I peptide cargo for immune evasion. Immunity. 20(1):71-85 (2004); Browne, H et al. A complex between the MHC class I homologue encoded by human cytomegalovirus and beta 2 microglobulin. Nature. 347(6295):770-2 (1990); Fahnestock, M L et al. The MHC class I homolog encoded by human cytomegalovirus binds endogenous peptides. Immunity. 3(5):583-90 (1995)). For example, the UL18 gene of the CMV TR strain is 24005 to 25111 for GenBank Accession No. KF021605.1.

The human cytomegalovirus UL78 gene encodes a putative G protein-coupled receptor (Chee, M S et al. Analysis of the protein-coding content of the sequence of human cytomegalovirus strain AD169. Curr Top Microbiol Immunol. 1154:125-69 (1990)) and may also have a role in viral replication (Michel, D et al. The human cytomegalovirus UL78 gene is highly conserved among clinical isolates, but is dispensable for replication in fibroblasts and a renal artery organ-culture system. J Gen Virol. 86(Pt 2):297-306 (2005)). For example, the UL78 gene of the CMV TR strain is 114247 to 115542 for GenBank Accession No. KF021605.1.

In some embodiments, the recombinant CMV vector expresses UL128 or UL130, or orthologs thereof. In some embodiments, the recombinant CMV vector expresses UL146 and UL147, or orthologs thereof. In some embodiments, the recombinant CMV vector expresses UL128, UL130, UL146, and UL147.

In some embodiments, the recombinant CMV vector (e.g., a recombinant HCMV vector or a recombinant HCMV vector comprising a TR3 backbone) does not express UL128 or UL130, or orthologs thereof, due to the presence of a mutation in the nucleic acid sequences encoding UL128 and UL130, or the orthologs thereof. In some embodiments, the CMV vector is deficient for one or more of UL146, UL147, UL18, UL78, and UL82, and orthologs thereof, due to the presence of a mutation in the nucleic acid sequence encoding UL146, UL147, UL18, UL78, or UL82, or the ortholog thereof. In some embodiments, the CMV vector is deficient for US 11, and orthologs thereof, due to the presence of a mutation in the nucleic acid sequence encoding US11, or the ortholog thereof. In the aforementioned embodiments, the mutation or mutations may be any mutation that results in a lack of expression of active proteins. Such mutations include, for example, point mutations, frameshift mutations, deletions of less than all of the sequence that encodes the protein (truncation mutations), or deletions of all of the nucleic acid sequence that encodes the protein. In some embodiments, the recombinant CMV vector (e.g., a recombinant HCMV vector or a recombinant HCMV vector comprising a TR3 backbone) expresses UL40 and US28, or orthologs thereof.

In some embodiments, the recombinant CMV vector (e.g., a recombinant HCMV vector or a recombinant HCMV vector comprising a TR3 backbone) does not express UL78, UL128, or UL130, or orthologs thereof, due to the presence of a mutation in the nucleic acid sequences encoding UL78, UL128, and UL130, or the orthologs thereof. In some further embodiments, a nucleic acid molecule encoding a fusion protein as disclosed herein is replaces UL78. In some further embodiments, the fusion protein is operably linked to and is expressed by the UL78 promoter.

In some embodiments, the recombinant CMV vector (e.g., a recombinant HCMV vector or a recombinant HCMV vector comprising a TR3 backbone) does not express UL78, UL128, or UL130, or orthologs thereof, due to the presence of a mutation in the nucleic acid sequences encoding UL78, UL128, and UL130, or the orthologs thereof. In some further embodiments, the recombinant CMV vector expresses UL18, UL82, UL146, and UL147, or orthologs thereof. In some further embodiments, a nucleic acid molecule encoding a fusion protein as disclosed herein is replaces UL78. In some further embodiments, the fusion protein is operably linked to and is expressed by the UL78 promoter.

In some embodiments, the recombinant CMV vector (e.g., a recombinant HCMV vector or a recombinant HCMV vector comprising a TR3 backbone) does not express UL82, UL128, or UL130, or orthologs thereof, due to the presence of a mutation in the nucleic acid sequences encoding UL82, UL128, and UL130, or the orthologs thereof. In some further embodiments, a nucleic acid molecule encoding a fusion protein disclosed herein is replaces UL82. In some further embodiments, the fusion protein is operably linked to and is expressed by the UL82 promoter.

A challenge for manufacturing HCMV vectors having desirable properties for vaccines is that the vectors are often designed to have reduced viral replication or growth. For example, some live attenuated HCMV-HIV vaccine vectors are engineered to be growth deficient by deletion of the HCMV gene UL82 (which encodes the tegument protein pp71), resulting in lower viral yield. pp71 is important for wild type HCMV infection because this tegument protein is translocated to the nucleus where it suppresses cellular Daxx function, thus allowing CMV immediate-early (IE) gene expression that triggers the replication cycle. Some manufacturing processes rely on functional complementation using transient transfection of MRC-5 cells with an siRNA targeting Daxx, which mimics one of the functions of HCMV pp71. Another approach is to use transfection of a mRNA encoding pp71, to enable the host cell to express the essential viral gene. Transfection of a mRNA for expressing the essential viral gene may be able to provide all of the functions of the gene that are likely to enhance the infection process, such as cell cycle stimulation, efficient virion packaging, and virus stability. In addition, protein present late in infection has the potential to be packaged in the progeny virus, which could lower the required dose of the vaccine by more efficient first round infection and establishment of persistent infection. Accordingly, in some embodiments, the present disclosure provides a method of producing a recombinant CMV viral vector, comprising: (a) introducing a mRNA encoding a pp71 protein to a cell; (b) infecting the cell with a recombinant CMV; (c) incubating the cell; and (d) collecting the recombinant CMV viral vector. In some embodiments, the nucleic acid encoding a pp71 protein is delivered to the cell using transfection. In some embodiments, the cell is a MRC-5 cell. In some embodiments, the recombinant CMV is a recombinant HCMV as described herein (e.g., a recombinant HCMV vector derived from a TR3 backbone). In some embodiments, the recombinant CMV and recombinant CMV viral vector comprises a nucleic acid encoding a heterologous pathogen-specific antigen, such as a Mtb antigen as described herein. A CMV viral vector made by such a method is also within the scope of the disclosure.

In some embodiments, the recombinant CMV vector (e.g., a recombinant HCMV vector or a recombinant HCMV vector comprising a TR3 backbone) comprises a nucleic acid sequence encoding a microRNA (miRNA) recognition element (MRE). In some embodiments, the HCMV vector comprises a nucleic acid sequence encoding an MRE that contains target sites for microRNAs expressed in endothelial cells. Examples of miRNAs expressed in endothelial cells are miR126, miR-126-3p, miR-130a, miR-210, miR-221/222, miR-378, miR-296, and miR-328. In some embodiments, the recombinant CMV vector (e.g., a recombinant HCMV vector or a recombinant HCMV vector comprising a TR3 backbone) comprises a nucleic acid sequence encoding an MRE that contains target sites for microRNAs expressed in myeloid cells. Examples of miRNAs expressed in myeloid cells are miR-142-3p, miR-223, miR-27a, miR-652, miR-155, miR-146a, miR-132, miR-21, miR-124, and miR-125.

MREs that may be included in the vectors disclosed herein may be any miRNA recognition element that silences expression in the presence of a miRNA expressed by endothelial cells or a miRNA expressed by myeloid cells. Such an MRE may be the exact complement of a miRNA. Alternatively, other sequences may be used as MREs for a given miRNA. For example, MREs may be predicted from sequences using publicly available data bases. In one example, the miRNA may be searched on the website microRNA.org (www.microrna.org). In turn, a list of mRNA targets of the miRNA will be listed. For each listed target on the page, ‘alignment details’ may be accessed and putative MREs accessed. One of ordinary skill in the art may select a validated, putative, or mutated MRE sequence from the literature that would be predicted to induce silencing in the presence of a miRNA expressed in a myeloid cell such as a macrophage. One example involves the above referenced website. The person of ordinary skill in the art may then obtain an expression construct whereby a reporter gene (such as a fluorescent protein, enzyme, or other reporter gene) has expression driven by a promoter such as a constitutively active promoter or cell specific promoter. The MRE sequence may then be introduced into the expression construct. The expression construct may be transfected into an appropriate cell, and the cell transfected with the miRNA of interest. A lack of expression of the reporter gene indicates that the MRE silences gene expression in the presence of the miRNA.

In some embodiments, the CMV vector comprises a nucleic acid sequence that does not encode any MREs.

In some embodiments, the CMV vectors described herein contain mutations that may prevent host to host spread, thereby rendering the virus unable to infect immunocompromised or other subjects that could face complications as a result of CMV infection. The CMV vectors described herein may also contain mutations that result in the presentation of immunodominant and nonimmunodominant epitopes as well as non-canonical MHC restriction. However, in some embodiments, mutations in the CMV vectors described herein do not affect the ability of the vector to reinfect a subject that has been previously infected with CMV. Such CMV mutations are described in, for example, U.S. Patent Application Publication Nos. US2013/0136768A1, US2013/0142823A1; US2014/0141038A1; and International Application Publication No. WO2014/138209A1, which are incorporated by reference herein for teachings related to these mutations.

The CMV vectors disclosed herein may be prepared by inserting DNA comprising a sequence that encodes the Mtb antigen (e.g., a fusion protein as disclosed herein) into an essential or non-essential region of the CMV genome. The method may further comprise deleting one or more regions from the CMV genome. The method may comprise in vivo recombination. Thus, the method may comprise transfecting a cell with CMV DNA in a cell-compatible medium in the presence of donor DNA comprising the heterologous DNA flanked by DNA sequences homologous with portions of the CMV genome, whereby the heterologous DNA is introduced into the genome of the CMV, and optionally then recovering CMV modified by the in vivo recombination. The method may also comprise cleaving CMV DNA to obtain cleaved CMV DNA, ligating the heterologous DNA to the cleaved CMV DNA to obtain hybrid CMV-heterologous DNA, transfecting a cell with the hybrid CMV-heterologous DNA, and optionally then recovering CMV modified by the presence of the heterologous DNA Since in vivo recombination is comprehended, the method accordingly also provides a plasmid comprising donor DNA not naturally occurring in CMV encoding a polypeptide foreign to CMV, the donor DNA is within a segment of CMV DNA that would otherwise be co-linear with an essential or non-essential region of the CMV genome such that DNA from an essential or nonessential region of CMV is flanking the donor DNA The heterologous DNA may be inserted into CMV to generate the recombinant CMV in any orientation that yields stable integration of that DNA, and expression thereof, when desired.

The DNA encoding the Mtb antigen (e.g., a fusion protein as disclosed herein) in the recombinant CMV vector may also include a promoter. The promoter may be from any source such as a herpes virus, including an endogenous cytomegalovirus (CMV) promoter, such as a human CMV (HCMV), rhesus macaque CMV (RhCMV), murine, or other CMV promoter. The promoter may also be a nonviral promoter such as the EF1α promoter. The promoter may be a truncated transcriptionally active promoter which comprises a region transactivated with a transactivating protein provided by the virus and the minimal promoter region of the full-length promoter from which the truncated transcriptionally active promoter is derived. The promoter may be composed of an association of DNA sequences corresponding to the minimal promoter and upstream regulatory sequences. A minimal promoter is composed of the CAP site plus ATA box (minimum sequences for basic level of transcription; unregulated level of transcription); “upstream regulatory sequences” are composed of the upstream element(s) and enhancer sequence(s). Further, the term “truncated” indicates that the full-length promoter is not completely present, i.e., that some portion of the full-length promoter has been removed. The truncated promoter may be derived from a herpesvirus such as MCMV or HCMV, e.g., HCMV-IE or MCMV-IE. There may be up to a 40% and even up to a 90% reduction in size, from a full-length promoter, based upon base pairs. The promoter may also be a modified non-viral promoter. As to HCMV promoters, reference is made to U.S. Pat. Nos. 5,168,062 and 5,385,839. As to transfecting cells with plasmid DNA for expression therefrom, reference is made to Felgner, J H et al. (Enhanced gene delivery and mechanism studies with a novel series of cationic lipid formulations. J Biol. Chem. 269, 2550-2561 (1994)). As to direct injection of plasmid DNA as a simple and effective method of vaccination against a variety of infectious diseases reference is made to Ulmer, J B et al. (Heterologous protection against influenza by injection of DNA encoding a viral protein. Science 259, 1745-1749 (1993)). It is therefore within the scope of this disclosure that the vector may be used by the direct injection of vector DNA. Also disclosed is an expression cassette that may be inserted into a recombinant virus or plasmid comprising a truncated transcriptionally active promoter. The expression cassette may further include a functional truncated polyadenylation signal; for instance an SV40 polyadenylation signal which is truncated, yet functional. A truncated polyadenylation signal addresses the insert size limit problems of recombinant viruses such as CMV. The expression cassette may also include heterologous DNA with respect to the virus or system into which it is inserted; and that DNA may be heterologous DNA as described herein.

It is noted that the DNA comprising the sequence encoding the fusion protein may itself include a promoter for driving expression in the CMV vector or the DNA may be limited to the coding DNA of the fusion protein. This construct may be placed in such an orientation relative to an endogenous CMV promoter that it is operably linked to the promoter and is thereby expressed. Further, multiple copies of DNA encoding the fusion protein or use of a strong or early promoter or early and late promoter, or any combination thereof, may be done so as to amplify or increase expression. Thus, the DNA encoding the fusion protein may be suitably positioned with respect to a CMV endogenous promoter, or those promoters may be translocated to be inserted at another location together with the DNA encoding the fusion protein. Nucleic acids encoding more than one fusion protein, or a fusion protein and additional antigens, may be packaged in the CMV vector.

In some embodiments, the present disclosure provides a recombinant HCMV vector comprising a nucleic acid sequence having at least 90%, 91%, 92%, 93%, 94%, 95%, 96%, 97%, 98%, 99%, or 100% identity to the nucleic acid sequence according to SEQ ID NO:44. In some embodiments, the recombinant HCMV vector comprises the nucleic acid sequence according to SEQ ID NO:44. In some embodiments, the recombinant HCMV vector consists of the nucleic acid sequence according to SEQ ID NO:44.

### IV. Pharmaceutical Compositions

The present disclosure provides, in some embodiments, a pharmaceutical composition (e.g., an immunogenic or vaccine composition) comprising a fusion protein as described herein and a pharmaceutically acceptable carrier or diluent. The present disclosure also provides, in some embodiments, a pharmaceutical composition (e.g., an immunogenic or vaccine composition) comprising vector encoding a fusion protein as described herein (e.g., a recombinant CMV vectors disclosed herein). An immunogenic or vaccine composition containing the recombinant CMV virus or vector (or an expression product thereof) elicits an immunological response (local or systemic). The response can, but need not be, protective. In other words, an immunogenic or vaccine composition elicits a local or systemic protective or therapeutic response.

Such pharmaceutical compositions may be prepared in accordance with standard techniques well known to those skilled in the pharmaceutical arts. Such compositions may be administered in dosages and by techniques well known to those skilled in the medical arts taking into consideration such factors as the breed or species, age, sex, weight, and condition of the particular patient, and the route of administration. The compositions may be administered alone, or may be co-administered or sequentially administered with other proteins or peptides, with other vectors (e.g., other CMV vectors), or with other immunological, antigenic or vaccine or therapeutic compositions. Such other compositions may include purified native antigens or epitopes or antigens or epitopes from the expression by a recombinant CMV or another vector system.

Pharmaceutical compositions as disclosed herein may be formulated so as to be used in any administration procedure known in the art. Such pharmaceutical compositions may be via a parenteral route (intradermal, intraperitoneal, intramuscular, subcutaneous, intravenous, or others). The administration may also be via a mucosal route, e.g., oral, nasal, genital, etc.

Examples of compositions include liquid preparations for orifice, e.g., oral, nasal, anal, genital, e.g., vaginal, etc., administration such as suspensions, syrups or elixirs; and, preparations for parenteral, subcutaneous, intraperitoneal, intradermal, intramuscular or intravenous administration (e.g., injectable administration) such as sterile suspensions or emulsions. In such compositions the recombinant may be in admixture with a suitable carrier, diluent, or excipient such as sterile water, physiological saline, glucose, trehalose, or the like.

Pharmaceutical compositions disclosed herein typically may comprise or contain an adjuvant and an amount of the antigen (e.g., fusion protein) or vector encoding the antigen, to elicit the desired response. In human applications, alum (aluminum phosphate or aluminum hydroxide) is a typical adjuvant. Saponin and its purified component Quil A, Freund's complete adjuvant and other adjuvants used in research and veterinary applications have toxicities which limit their potential use in human vaccines. Chemically defined preparations such as muramyl dipeptide, monophosphoryllipid A, phospholipid conjugates such as those described by Goodman-Snitkoff, G. et al. (Role of intrastructural/intermolecular help in immunization with peptide-phospholipid complexes. J Immunol. 147, 410-415 (1991)), encapsulation of the protein within a proteoliposome as described by Miller, M D et al. (Vaccination of rhesus monkeys with synthetic peptide in a fusogenic proteoliposome elicits simian immunodeficiency virus-specific CD8+ cytotoxic T lymphocytes. J Exp. Med. 176, 1739-1744 (1992)), and encapsulation of the protein in lipid vesicles such as Novasome lipid vesicles (Micro Vescular Systems, Inc., Nashua, N.H.) may also be used.

The composition may be packaged in a single dosage form for immunization by parenteral (e.g., intramuscular, intradermal or subcutaneous) administration or orifice administration, e.g., perlingual (e.g., oral), intragastric, mucosal including intraoral, intraanal, intravaginal, and the like administration. The effective dosage and route of administration are determined by the nature of the composition, by the nature of the expression product, by expression level if a vector is directly used, and by known factors, such as breed or species, age, sex, weight, condition, and nature of the subject, as well as LD50 and other screening procedures which are known and do not require undue experimentation. Dosages of expressed product may range from a few to a few hundred micrograms, e.g., 5 to 500 μg. The antigen or vector encoding the antigen may be administered in any suitable amount to achieve expression at these dosage levels. In nonlimiting examples: a CMV vector encoding the fusion protein disclosed herein may be administered in an amount of at least 102 pfu; or in a range from about 102 pfu to about 107 pfu. Other suitable carriers or diluents may be water or a buffered saline, with or without a preservative. The composition may be lyophilized for resuspension at the time of administration or may be in solution.

### V. Methods of Treatment and Other Uses

The fusion proteins and vectors (such as recombinant CMV vectors) disclosed herein may be used in methods of inducing an immunological or immune response in a subject comprising administering to the subject a composition comprising the fusion protein, the vector, or the recombinant CMV virus or vector and a pharmaceutically acceptable carrier or diluent.

As used herein, the term “subject” refers to a living multi-cellular vertebrate organisms, a category that includes both human and non-human mammals. The subject may be an animal, such as a mammal, including any mammal that can be infected with Mycobacterium tuberculosis, e.g., a primate (such as a human, a non-human primate, e.g., a monkey, or a chimpanzee), or an animal that is considered an acceptable clinical model of tuberculosis infection.

In some embodiments, the subject is human. In further embodiments, the subject is an adult 18 years old or older. In still further embodiments, the subject is an adolescent aged 13 to 17 years old.

In some embodiments, the subject resides in a geographical location where tuberculosis is not endemic. In some embodiments, the subject resides in a geographical location where tuberculosis is endemic (e.g., South Africa). As used herein, “endemic” is used to describe a disease that is constantly present in a certain geographic area or in a certain group of people.

In some embodiments the subject has tested positive for CMV. In some embodiments the patient has tested negative for CMV. CMV testing refers to assays that determine the presence of the virus in the urine, saliva, blood, sputum, or other body fluids. Non-limiting examples of CMV tests include polymerase chain reaction (PCR), e.g., a CMV-salivary PCR test.

In some embodiments the subject has a positive result from an interferon-y release assay (IGRA). In some embodiments the subject has a negative result from an interferon-y release assay. An interferon-y release assay is used to diagnose tuberculosis. T lymphocytes will release interferon-y upon exposure to specific Mycobacterium tuberculosis antigens indicating previous exposure to the Mycobacterium tuberculosis antigens tested.

In some embodiments the subject has tested positive for CMV and has a positive result from an interferon-y release assay. In some embodiments the subject has tested negative for CMV and has a negative result from an interferon-y release assay. In some embodiments the subject has tested negative for CMV and has a positive result from an interferon-y release assay. In some embodiments the subject has tested positive for CMV and has a negative result from an interferon-y release assay. In further embodiments the subject has been previously administered the bacille Calmette-Guerin vaccine (BCG). In still further embodiments, the subject has tested positive for CMV, has a negative result from an interferon-y release assay, and has additionally been previously administered the bacille Calmette-Guerin vaccine (BCG).

In some embodiments, the subject is HIV positive. In further embodiments, the subject is HIV positive and currently on anti-retroviral therapy (ART).

As used herein, the term “treatment” refers to an intervention that ameliorates a sign or symptom of a disease or pathological condition. As used herein, the terms “treatment”, “treat”, and “treating,” with reference to a disease, pathological condition or symptom, also refers to any observable beneficial effect of the treatment. The beneficial effect may be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

As used herein, the terms “preventing” or “prevention” refer to the failure to develop a disease, disorder, or condition, or the reduction in the development of a sign or symptom associated with such a disease, disorder, or condition (e.g., by a clinically relevant amount), or the exhibition of delayed signs or symptoms delayed (e.g., by days, weeks, months, or years). Prevention may require the administration of more than one dose.

As used herein, the term “effective amount” refers to an amount of an agent (e.g., a fusion protein or a vector encoding a fusion protein), that is sufficient to generate a desired response, such as reduce or eliminate a sign or symptom of a condition or disease or induce an immune response to an antigen. In some examples, an “effective amount” is one that treats (including prophylaxis) one or more symptoms and/or underlying causes of any of a disorder or disease. An effective amount may be a therapeutically effective amount, including an amount that prevents one or more signs or symptoms of a particular disease or condition from developing, such as one or more signs or symptoms associated with infectious disease or cancer.

The disclosed fusion proteins or vectors may be administered in vivo, for example where the aim is to produce an immunogenic response, including a CD4+ T cell/immune and/or a CD8+ T cell/immune. For example, in some examples it may be desired to use the disclosed fusion proteins or vectors in a laboratory animal, such as rhesus macaques for preclinical testing of immunogenic compositions and vaccines using RhCMV. In other examples, it will be desirable to use the fusion proteins or vectors in human subjects, such as in clinical trials and for actual clinical use of the immunogenic compositions using HCMV.

For such in vivo applications the disclosed fusion proteins or vectors may be administered as a component of an immunogenic or pharmaceutical composition further comprising a pharmaceutically acceptable carrier. In some embodiments, the immunogenic compositions of the disclosure are useful to stimulate an immune response against the fusion protein, and may be used as one or more components of a prophylactic or therapeutic vaccine. The nucleic acids and vectors of the disclosure are particularly useful for providing genetic vaccines, i.e., vaccines for delivering the nucleic acids encoding the antigens of the disclosure to a subject, such as a human, such that the antigens are then expressed in the subject to elicit an immune response.

Immunization schedules (or regimens) are well known for animals (including humans) and may be readily determined for the particular subject and immunogenic composition. Hence, the immunogens may be administered one or more times to the subject. Preferably, there is a set time interval between separate administrations of the immunogenic composition. While this interval varies for every subject, typically it ranges from 10 days to several weeks, and is often 2, 4, 6, or 8 weeks. For humans, the interval is typically from 2 to 6 weeks. In a particularly advantageous embodiment of the present disclosure, the interval is longer, advantageously about 10 weeks, 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks, 54 weeks, 56 weeks, 58 weeks, 60 weeks, 62 weeks, 64 weeks, 66 weeks, 68 weeks, or 70 weeks. The immunization regimes typically have from 1 to 6 administrations of the immunogenic composition, but may have as few as one or two or four. The methods of inducing an immune response may also include administration of an adjuvant with the immunogens. In some instances, annual, biannual, or other long interval (5-10 years) booster immunization may supplement the initial immunization protocol. The present methods also include a variety of prime-boost regimens. In these methods, one or more priming immunizations are followed by one or more boosting immunizations. The actual immunogenic composition may be the same or different for each immunization and the type of immunogenic composition (e.g., containing protein or expression vector), the route, and formulation of the immunogens may also be varied. For example, if an expression vector is used for the priming and boosting steps, it may either be of the same or different type (e.g., DNA or bacterial or viral expression vector). One useful prime-boost regimen provides for two priming immunizations, four weeks apart, followed by two boosting immunizations at 4 and 8 weeks after the last priming immunization. It should also be readily apparent to one of skill in the art that there are several permutations and combinations that are encompassed using the DNA, bacterial, and viral expression vectors of the disclosure to provide priming and boosting regimens. CMV vectors may be used repeatedly while expressing different antigens derived from different pathogens.

Accordingly, the present disclosure provides, in some embodiments, a method of generating an immune response in a subject, comprising administering to the subject any of the aforementioned fusion proteins, nucleic acids, vectors, or compositions. Also provided herein is the use of any of the aforementioned fusion proteins, nucleic acids, vectors, or compositions in the manufacture of a medicament for use in generating an immune response in a subject.

In some embodiments, the present disclosure provides a method of treating or preventing tuberculosis in a subject, comprising administering to the subject a fusion protein, nucleic acid, vector, or composition described herein. In some embodiments, a fusion protein, nucleic acid, vector, or composition described herein is used in the manufacture of a medicament for use in treating or preventing tuberculosis in a subject. In some embodiments, the tuberculosis is a latent tuberculosis infection. In some embodiments, the present disclosure provides a method of preventing tuberculosis disease in a subject. In further embodiments, the subject has had a positive result from an interferon-y release assay. In some embodiments, the present disclosure provides a method of preventing Mycobacterium tuberculosis infection. In further embodiments, the subject has had a negative result from an interferon-γ release assay.

In some embodiments, the present disclosure provides a method of preventing recurrence of tuberculosis and/or M. tuberculosis infection in a subject. In further embodiments, the prevention of recurrence occurs after a previous treatment for tuberculosis.

In some embodiments, the fusion proteins and vectors (such as recombinant CMV vectors) disclosed herein are administered previously to, concurrently with, or subsequently to a second tuberculosis treatment. In some embodiments, the fusion proteins and vectors disclosed herein are adjunct to the second treatment. In some embodiments, the subject receiving adjunct treatment is infected with drug-resistant M. tuberculosis.

In some embodiments, the present disclosure provides a method of preventing pulmonary tuberculosis in a subject.

In some of the aforementioned methods, uses, or compositions for use, the vector is a CMV vector and the CMV vector is administered in an amount effective to elicit a CD4+ T cell response to a Mtb antigen. In some embodiments, at least 10% of the CD4+ T cells elicited by the recombinant HCMV vector are restricted by MHC-II or an ortholog thereof. In some further embodiments, at least 20%, at least 30%, at least 40%, at least 50%, at least 60%, at least 75%, at least 80%, at least 85%, at least 90%, or at least 95% of the CD4+ T cells elicited by the recombinant HCMV vector are restricted by MHC-II or an ortholog thereof.

In some of the aforementioned methods, uses, or compositions for use, the vector is a CMV vector and the CMV vector is administered in an amount effective to elicit a CD8+ T cell response to a Mtb antigen. In some embodiments, at least 10% of the CD8+ T cells elicited by the CMV vector are restricted by MHC-Ia or an ortholog thereof. In some further embodiments, at least 20%, at least 30%, at least 40%, at least 50%, at least 60%, at least 75%, at least 80%, at least 85%, at least 90%, or at least 95% of the CD8+ T cells elicited by the CMV vector are restricted by MHC-Ia or an ortholog thereof.

In some further aspects, the present disclosure provides a method of generating CD4+ T cells that recognize MHC-II/peptide complexes by administering a CMV vector encoding a fusion protein described herein. In some embodiments, the method comprises:
(a) administering to a first subject a CMV vector described herein in an amount effective to generate a set of CD4+ T cells that recognize MHC-II/peptide complexes;
(b) identifying a first CD4+ TCR from the set of CD4+ T cells, wherein the first CD4+ TCR recognizes a MHC-II/fusion protein-derived peptide complex;
(c) isolating one or more CD4+ T cells from a second subject; and
(d) transfecting the one or more CD4+ T cells isolated from the second subject with an expression vector, wherein the expression vector comprises a nucleic acid sequence encoding a second CD4+ TCR and a promoter operably linked to the nucleic acid sequence encoding the second CD4+ TCR, wherein the second CD4+ TCR comprises CDR3α and CDR3β of the first CD4+ TCR, thereby generating one or more CD4+ T cells that recognize MHC-II/peptide complexes.

In some embodiments, the method comprises:
(a) identifying a first CD4+ TCR from a set of CD4+ T cells, wherein the set of CD4+ T cells are isolated from a subject that has been administered a CMV vector described herein, and wherein the first CD4+ TCR recognizes a MHC-II/fusion protein-derived peptide complex;
(b) isolating one or more CD4+ T cells from a second subject; and
(c) transfecting the one or more CD4+ T cells isolated from the second subject with an expression vector, wherein the expression vector comprises a nucleic acid sequence encoding a second CD4+ TCR and a promoter operably linked to the nucleic acid sequence encoding the second CD4+ TCR, wherein the second CD4+ TCR comprises CDR3α and CDR3β of the first CD4+ TCR, thereby generating one or more TCR-transgenic CD4+ T cells that recognize MHC-II/peptide complexes.

In some further aspects, the present disclosure provides a method of generating CD8+ T cells that recognize MHC-Ia/peptide complexes by administering a CMV vector encoding a fusion protein described herein. In some embodiments, the method comprises:
(a) administering to a first subject a CMV vector disclosed herein in an amount effective to generate a set of CD8+ T cells that recognize MHC-Ia/peptide complexes;
(b) identifying a first CD8+ TCR from the set of CD8+ T cells, wherein the first CD8+ TCR recognizes a MHC-Ia/fusion protein-derived peptide complex;
(c) isolating one or more CD8+ T cells from a second subject; and
(d) transfecting the one or more CD8+ T cells isolated from the second subject with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize MHC-Ia/peptide complexes.

In some embodiments, the method comprises:
(a) identifying a first CD8+ TCR from a set of CD8+ T cells, wherein the set of CD8+ T cells are isolated from a subject that has been administered a CMV vector disclosed herein, and wherein the first CD8+ TCR recognizes a MHC-Ia/fusion protein-derived peptide complex;
(b) isolating one or more CD8+ T cells from a second subject; and
(c) transfecting the one or more CD8+ T cells isolated from the second subject with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more TCR-transgenic CD8+ T cells that recognize MHC-Ia/peptide complexes.

In some embodiments of the methods of generating T cells, wherein the first CD4+ TCR or the first CD8+ TCR is identified by DNA or RNA sequencing. In some embodiments, wherein the nucleic acid sequence encoding the second CD4+ TCR or the nucleic acid sequence encoding the second CD4+ TCR is identical to the nucleic acid sequence encoding the first CD4+ TCR or the first CD8+ TCR. In some embodiments, the first and second subjects are human.

The present disclosure also provides a CD4+ T cell or CD8+ T cell generated by the aforementioned methods. In some further embodiments, the CD4+ or CD8+ T cell is used in a method of treating or preventing a disease in a subject. The CD4+ or CD8+ T cell may be used in still further embodiments in the manufacture of a medicament for use in treating or preventing a disease in a subject.

### VI. Example Embodiments

In some embodiments, the present disclosure provides:

1. A fusion protein comprising or consisting of:
(a) Ag85A, ESAT-6, Rv3407, Rv2626c, Ra12, TbH9, Ra35, and RpfD, or fragments thereof;
(b) Ag85A-ESAT-6-Rv3407-Rv2626c-Ra12-TbH9-Ra35-RpfD;
(c) an amino acid sequence having 90%, 91%, 92%, 93%, 94%, 95%, 96%, 97%, 98%, 99%, or 100% identity to the amino acid sequence according to SEQ ID NO:42;
(d) the amino acid sequence according to SEQ ID NO:42;
(e) Ag85A, ESAT-6, Rv3407, Rv2626c, RpfA, RpfD, Ra12, TbH9, and Ra35, or fragments thereof;
(f) Ag85A-ESAT-6-Rv3407-Rv2626c-RpfA-RpfD-Ra12-TbH9-Ra35;
(g) (i) an amino acid sequence having 90%, 91%, 92%, 93%, 94%, 95%, 96%, 97%, 98%, 99%, or 100% identity to the amino acid sequence according to any one of SEQ ID NOs:9-10; and (ii) an amino acid sequence having 90%, 91%, 92%, 93%, 94%, 95%, 96%, 97%, 98%, 99%, or 100% identity to the amino acid sequence according to any one of SEQ ID NOs:18-22;
(h) (i) an amino acid sequence having 90%, 91%, 92%, 93%, 94%, 95%, 96%, 97%, 98%, 99%, or 100% identity to the amino acid sequence according to SEQ ID NO:10; and (ii) an amino acid sequence having 90%, 91%, 92%, 93%, 94%, 95%, 96%, 97%, 98%, 99%, or 100% identity to the amino acid sequence according to SEQ ID NO:19;
(i) (i) an amino acid sequence having 90%, 91%, 92%, 93%, 94%, 95%, 96%, 97%, 98%, 99%, or 100% identity to the amino acid sequence according to SEQ ID NO:10; and (ii) an amino acid sequence having 90%, 91%, 92%, 93%, 94%, 95%, 96%, 97%, 98%, 99%, or 100% identity to the amino acid sequence according to SEQ ID NO:22;
(j) (i) the amino acid sequence according to SEQ ID NO:10; and (ii) the amino acid sequence according to SEQ ID NO:19;
(k) (i) the amino acid sequence according to SEQ ID NO:10; and (ii) the amino acid sequence according to SEQ ID NO:22;
(l) an amino acid sequence having 90%, 91%, 92%, 93%, 94%, 95%, 96%, 97%, 98%, 99%, or 100% identity to the amino acid sequence according to SEQ ID NO:27;
(m) an amino acid sequence having 90%, 91%, 92%, 93%, 94%, 95%, 96%, 97%, 98%, 99%, or 100% identity to the amino acid sequence according to SEQ ID NO:28;
(n) an amino acid sequence having 90%, 91%, 92%, 93%, 94%, 95%, 96%, 97%, 98%, 99%, or 100% identity to the amino acid sequence according to SEQ ID NO:29;
(o) an amino acid sequence having 90%, 91%, 92%, 93%, 94%, 95%, 96%, 97%, 98%, 99%, or 100% identity to the amino acid sequence according to SEQ ID NO:30;
(p) the amino acid sequence according to SEQ ID NO:27;
(q) the amino acid sequence according to SEQ ID NO:28;
(r) the amino acid sequence according to SEQ ID NO:29;
(s) the amino acid sequence according to SEQ ID NO:30;
(t) Ag85A, ESAT-6, Rv3407, Rv2626c, RpfA, RpfD, and TbH9, or fragments thereof;
(u) Ag85A-ESAT-6-Rv3407-Rv2626c-RpfA-RpfD-TbH9;
(v) (i) an amino acid sequence having 90%, 91%, 92%, 93%, 94%, 95%, 96%, 97%, 98%, 99%, or 100% identity to the amino acid sequence according to any one of SEQ ID NOs:9-10; and (ii) an amino acid sequence having 90%, 91%, 92%, 93%, 94%, 95%, 96%, 97%, 98%, 99%, or 100% identity to the amino acid sequence according to SEQ ID NO:24;
(w) (i) an amino acid sequence having 90%, 91%, 92%, 93%, 94%, 95%, 96%, 97%, 98%, 99%, or 100% identity to the amino acid sequence according to SEQ ID NO:10; and (ii) an amino acid sequence having 90%, 91%, 92%, 93%, 94%, 95%, 96%, 97%, 98%, 99%, or 100% identity to the amino acid sequence according to SEQ ID NO:24;
(x) (i) the amino acid sequence according to SEQ ID NO:10; and (ii) the amino acid sequence according to SEQ ID NO:24;
(y) an amino acid sequence having 90%, 91%, 92%, 93%, 94%, 95%, 96%, 97%, 98%, 99%, or 100% identity to the amino acid sequence according to SEQ ID NO:31;
(z) an amino acid sequence having 90%, 91%, 92%, 93%, 94%, 95%, 96%, 97%, 98%, 99%, or 100% identity to the amino acid sequence according to SEQ ID NO:32;
(aa) the amino acid sequence according to SEQ ID NO:31;
(bb) the amino acid sequence according to SEQ ID NO:32;
(cc) Ag85A, ESAT-6, Rv3407, Rv2626c, RpfD, Ra12, TbH9, and Ra35, or fragments thereof;
(dd) Ag85A-ESAT-6-Rv3407-Rv2626c-RpfD-Ra12-TbH9-Ra35;
(ee) (i) an amino acid sequence having 90%, 91%, 92%, 93%, 94%, 95%, 96%, 97%, 98%, 99%, or 100% identity to the amino acid sequence according to any one of SEQ ID NOs:1 and 11-12; (ii) an amino acid sequence having 90%, 91%, 92%, 93%, 94%, 95%, 96%, 97%, 98%, 99%, or 100% identity to the amino acid sequence according to SEQ ID NO:2 or 13; (iii) an amino acid sequence having 90%, 91%, 92%, 93%, 94%, 95%, 96%, 97%, 98%, 99%, or 100% identity to the amino acid sequence according to SEQ ID NO:3 or 14; (iv) an amino acid sequence having 90%, 91%, 92%, 93%, 94%, 95%, 96%, 97%, 98%, 99%, or 100% identity to the amino acid sequence according to SEQ ID NO:4 or 15; (v) an amino acid sequence having 90%, 91%, 92%, 93%, 94%, 95%, 96%, 97%, 98%, 99%, or 100% identity to the amino acid sequence according to SEQ ID NO:6 or 17; (vi) an amino acid sequence having 90%, 91%, 92%, 93%, 94%, 95%, 96%, 97%, 98%, 99%, or 100% identity to the amino acid sequence according to SEQ ID NO:23; (vii) an amino acid sequence having 90%, 91%, 92%, 93%, 94%, 95%, 96%, 97%, 98%, 99%, or 100% identity to the amino acid sequence according to SEQ ID NO:8 or 24; and (viii) an amino acid sequence having 90%, 91%, 92%, 93%, 94%, 95%, 96%, 97%, 98%, 99%, or 100% identity to the amino acid sequence according to any one of SEQ ID NOs:25-26;
(ff) an amino acid sequence having 90%, 91%, 92%, 93%, 94%, 95%, 96%, 97%, 98%, 99%, or 100% identity to the amino acid sequence according to SEQ ID NO:33;
(gg) an amino acid sequence having 90%, 91%, 92%, 93%, 94%, 95%, 96%, 97%, 98%, 99%, or 100% identity to the amino acid sequence according to SEQ ID NO:34;
(hh) an amino acid sequence having 90%, 91%, 92%, 93%, 94%, 95%, 96%, 97%, 98%, 99%, or 100% identity to the amino acid sequence according to SEQ ID NO:35;
(ii) an amino acid sequence having 90%, 91%, 92%, 93%, 94%, 95%, 96%, 97%, 98%, 99%, or 100% identity to the amino acid sequence according to SEQ ID NO:36;
(jj) the amino acid sequence according to SEQ ID NO:33;
(kk) the amino acid sequence according to SEQ ID NO:34;
(ll) the amino acid sequence according to SEQ ID NO:35;
(mm) the amino acid sequence according to SEQ ID NO:36;
(nn) Ag85A, ESAT-6, Rv3407, Rv2626c, RpfD, and TbH9, or fragments thereof;
(oo) Ag85A-ESAT-6-Rv3407-Rv2626c-RpfD-TbH9;
(pp) (i) an amino acid sequence having 90%, 91%, 92%, 93%, 94%, 95%, 96%, 97%, 98%, 99%, or 100% identity to the amino acid sequence according to any one of SEQ ID NOs:1 and 11-12; (ii) an amino acid sequence having 90%, 91%, 92%, 93%, 94%, 95%, 96%, 97%, 98%, 99%, or 100% identity to the amino acid sequence according to SEQ ID NO:2 or 13; (iii) an amino acid sequence having 90%, 91%, 92%, 93%, 94%, 95%, 96%, 97%, 98%, 99%, or 100% identity to the amino acid sequence according to SEQ ID NO:3 or 14; (iv) an amino acid sequence having 90%, 91%, 92%, 93%, 94%, 95%, 96%, 97%, 98%, 99%, or 100% identity to the amino acid sequence according to SEQ ID NO:4 or 15; (v) an amino acid sequence having 90%, 91%, 92%, 93%, 94%, 95%, 96%, 97%, 98%, 99%, or 100% identity to the amino acid sequence according to SEQ ID NO:6 or 17; and (vi) an amino acid sequence having 90%, 91%, 92%, 93%, 94%, 95%, 96%, 97%, 98%, 99%, or 100% identity to the amino acid sequence according to SEQ ID NO:8 or 24;
(qq) an amino acid sequence having 90%, 91%, 92%, 93%, 94%, 95%, 96%, 97%, 98%, 99%, or 100% identity to the amino acid sequence according to SEQ ID NO:37;
(rr) an amino acid sequence having 90%, 91%, 92%, 93%, 94%, 95%, 96%, 97%, 98%, 99%, or 100% identity to the amino acid sequence according to SEQ ID NO:38;
(ss) the amino acid sequence according to SEQ ID NO:37; or
(tt) the amino acid sequence according to SEQ ID NO:38.

2. A fusion protein encoded by a nucleic acid comprising a sequence having 90%, 91%, 92%, 93%, 94%, 95%, 96%, 97%, 98%, 99%, or 100% identity to the nucleic acid sequence according to SEQ ID NO:41.

3. A fusion protein encoded by a nucleic acid comprising the nucleic acid sequence according to SEQ ID NO:41.

4. The fusion protein of any one of embodiments 1-3, further comprising a poly-His tag.

5. The fusion protein of embodiment 4, wherein the poly-His tag comprises or consists of two to six His residues.

6. The fusion protein of any one of embodiments 1-5, wherein the poly-His tag is located at the N-terminus of the fusion protein.

7. The fusion protein of embodiment 6, wherein the poly-His tag is inserted after the initial Met residue.

8. The fusion protein of any of embodiments 1-7, further comprising a HA tag.

9. The fusion protein of embodiment 8, wherein the HA tag is located at the C-terminus of the fusion protein.

10. The fusion protein of any one of embodiments 1-9, wherein the fusion protein further comprises one or more linkers connecting one or more of Ag85A, ESAT-6, Rv3407, Rv2626c, RpfA, RpfD, Ra12, TbH9, and Ra35, wherein each of the one or more linkers comprises or consists of one or more amino acid residues.

11. A nucleic acid molecule encoding the fusion protein according to any one of embodiments 1-10.

12. A vector comprising the nucleic acid molecule of embodiment 11.

13. The vector of embodiment 12, further comprising a promoter, wherein a promoter is operably linked to the nucleic acid molecule encoding the fusion protein.

14. The vector of embodiment 12 or embodiment 13, wherein the vector is a viral vector.

15. The vector of embodiment 14, wherein the viral vector is a cytomegalovirus (CMV) vector.

16. A vector comprising a nucleic acid sequence having at least 90%, 91%, 92%, 93%, 94%, 95%, 96%, 97%, 98%, 99%, or 100% identity to the nucleic acid sequence according to SEQ ID NO:44.

17. A vector comprising the nucleic acid sequence according to SEQ ID NO:44.

18. A vector consisting essentially of the nucleic acid sequence according to SEQ ID NO:44.

19. A vector consisting of the nucleic acid sequence according to SEQ ID NO:44.

20. The vector of any one of embodiments 15-19, wherein the viral vector is a RhCMV vector, a HCMV vector, or a recombinant HCMV vector.

21. The vector of any one of embodiments 15-20, wherein the promoter is operably linked to the nucleic acid molecule encoding the fusion protein and the promoter is a UL78 promoter, or an ortholog thereof.

22. The vector of embodiment 21, wherein the nucleic acid molecule encoding the fusion protein replaces all or part of UL78.

23. The vector of embodiment 22, wherein the vector comprises a nucleic acid sequence having at least 90%, 91%, 92%, 93%, 94%, 95%, 96%, 97%, 98%, 99%, or 100% identity to the nucleic acid sequence according to SEQ ID NO:44.

24. The vector of embodiment 15 or embodiment 20, wherein the promoter is operably linked to the nucleic acid molecule encoding the fusion protein and the promoter is a UL82 promoter, or an ortholog thereof.

25. The vector of embodiment 24, wherein the nucleic acid molecule encoding the fusion protein replaces all or part of UL82

26. The vector of any one of embodiments 15-25, wherein the RhCMV vector or HCMV vector does not express UL128 or UL130, or orthologs thereof.

27. A recombinant HCMV vector comprising a TR3 backbone and a nucleic acid sequence encoding a heterologous antigen according to SEQ ID NO: 42, wherein:
(a) the vector does not express UL128 or UL130, or orthologs thereof,
(b) the vector comprises a nucleic acid sequence encoding UL146, UL147, UL18, and UL82 or orthologs thereof, and
(c) the heterologous antigen replaces all or part of UL78 and is operably linked to the UL78 promoter.

28. The recombinant HCMV vector of embodiment 27, wherein the recombinant HCMV vector comprises a nucleic acid sequence having at least 90%, 91%, 92%, 93%, 94%, 95%, 96%, 97%, 98%, 99%, or 100% identity to the nucleic acid sequence according to SEQ ID NO:44.

29. The vector of any one of embodiments 15-28, wherein the RhCMV or HCMV vector (i) comprises a nucleic acid sequence encoding UL146 and a nucleic acid sequence encoding UL147, or orthologs thereof, and (ii) does not express UL128 or UL130, or orthologs thereof.

30. The vector of any one of embodiments 26-29, wherein the vector does not express a UL128 protein or a UL130 protein, resulting from the presence of one or more mutations in the nucleic acid sequences encoding UL128 and UL130.

31. The vector of embodiment 30, wherein the mutation in the nucleic acid sequences encoding UL128 and UL130 is a point mutation, frameshift mutation, truncation mutation, or deletion of all of the nucleic acid sequences encoding the viral protein.

32. The vector of any one of embodiments 15-31, wherein the vector is a HCMV vector comprising a TR3 backbone.

33. The vector of any one of embodiments 15-32, wherein the vector further comprises a nucleic acid sequence encoding a microRNA (miRNA) recognition element (MRE), wherein the MRE contains a target site for a miRNA expressed in endothelial cells.

34. The vector of any one of embodiments 15-33, wherein the vector further comprises a nucleic acid sequence encoding a MRE, wherein the MRE contains a target site for a miRNA expressed in myeloid cells.

35. A pharmaceutical composition comprising (i) (a) the fusion protein of any one of embodiments 1-10, (b) the nucleic acid of embodiment 11, or (c) the vector of any one of embodiments 12-34; and (ii) a pharmaceutically acceptable carrier.

36. The pharmaceutical composition of embodiment 35, wherein the pharmaceutically acceptable carrier is a histidine trehalose (HT) buffer.

37. The pharmaceutical composition of embodiment 35 or 36, wherein the pharmaceutically acceptable carrier is a histidine trehalose (HT) buffer comprising about 20 mM L-histidine and about 10% (w/v) trehalose.

38. The pharmaceutical composition of any one of embodiments 35-37, wherein the pharmaceutically acceptable carrier is a histidine trehalose (HT) buffer comprising 20 mM L-histidine and 10% (w/v) trehalose.

39. The pharmaceutical composition of any one of embodiments 35-38, wherein the pharmaceutically acceptable carrier is a histidine trehalose (HT) buffer having a pH of 7.2 comprising 20 mM L-histidine and 10% (w/v) trehalose.

40. An immunogenic composition comprising (i) (a) the fusion protein of any one of embodiments 1-10, (b) the nucleic acid of embodiment 11, or (c) the vector of any one of embodiments 12-34; and (ii) a pharmaceutically acceptable carrier.

41. A method of generating an immune response in a subject, comprising administering to the subject the fusion protein, nucleic acid, vector, or composition of any one of embodiments 1-40.

42. Use of the fusion protein, nucleic acid, vector, or composition of any one of embodiments 1-40 in the manufacture of a medicament for use in generating an immune response in a subject.

43. The fusion protein, nucleic acid, vector, or composition of any one of embodiments 1-40 for use in generating an immune response in a subject.

44. A method of treating or preventing tuberculosis in a subject, comprising administering to the subject the fusion protein, nucleic acid, vector, or composition of any one of embodiments 1-40.

45. Use of the fusion protein, nucleic acid, vector, or composition of any one of embodiments 1-40 in the manufacture of a medicament for use in treating or preventing tuberculosis in a subject.

46. The fusion protein, nucleic acid, vector, or composition of any one of embodiments 1-40 for use in treating or preventing tuberculosis in a subject.

47. The fusion protein, nucleic acid, vector, or composition of any one of embodiments 1-40 for use in treating or preventing tuberculosis in a subject, wherein the subject is CMV positive.

48. The fusion protein, nucleic acid, vector, or composition of any one of embodiments 1-40 for use in treating or preventing tuberculosis in a subject, wherein the subject is CMV negative.

49. The fusion protein, nucleic acid, vector, or composition of any one of embodiments 1-40, 47, or 48 for use in treating or preventing tuberculosis in a subject, wherein the subject tests positive in an interferon-y release assay.

50. The fusion protein, nucleic acid, vector, or composition of any one of embodiments 1-40, 47, or 48 for use in treating or preventing tuberculosis in a subject, wherein the subject tests negative in an interferon-y release assay.

51. The fusion protein, nucleic acid, vector, or composition of any one of embodiments 1-40 or 47-50 for use in treating or preventing tuberculosis in a subject, wherein the subject has previously been administered bacille Calmette-Guerin vaccine (BCG).

52. The fusion protein, nucleic acid, vector, or composition of any one of embodiments 1-40 or 47-51 for use in treating or preventing tuberculosis in a subject, wherein the subject is HIV positive.

53. The fusion protein, nucleic acid, vector, or composition of any one of embodiments 1-40 or 47-52 for use in treating or preventing tuberculosis in a subject, wherein the subject is HIV positive and is currently taking anti-retroviral therapeutics.

54. The fusion protein, nucleic acid, vector, or composition of any one of embodiments 1-40 or 47-53 for use in treating or preventing tuberculosis in a subject, wherein the subject is administered a second therapy.

55. The method, use in manufacture, or use of any one of embodiments 44-54, wherein the tuberculosis is a latent tuberculosis infection.

56. The method, use in manufacture, or use of any one of embodiments 44-55, wherein the tuberculosis is a pulmonary tuberculosis infection.

57. The method, use in manufacture, or use of any one of embodiments 44-56, wherein the tuberculosis is a recurrent tuberculosis infection.

58. The method, use in manufacture, or use of any one of embodiments 41-57, wherein the vector is a CMV vector and the CMV vector is administered in an amount effective to elicit a CD4+ T cell response to a Mtb antigen.

59. The method, use in manufacture, or use of any one of embodiments 41-58, wherein the vector is a CMV vector and the CMV vector is administered in an amount of about 10² pfu to about 10⁷ pfu.

60. The method, use in manufacture, or use of embodiment 58 or embodiment 59, wherein at least 10% of the CD4+ T cells elicited by the recombinant HCMV vector are restricted by MHC-II or an ortholog thereof.

61. The method, use in manufacture, or use of embodiment 60, wherein at least 20%, at least 30%, at least 40%, at least 50%, at least 60%, at least 75%, at least 80%, at least 85%, at least 90%, or at least 95% of the CD4+ T cells elicited by the recombinant HCMV vector are restricted by MHC-II or an ortholog thereof.

62. The method, use in manufacture, or use of any one of embodiments 41-61, wherein the vector is a CMV vector and the CMV vector is administered in an amount effective to elicit a CD8+ T cell response to a Mtb antigen.

63. The method, use in manufacture, or vector or composition for use of embodiment 62, wherein at least 10% of the CD8+ T cells elicited by the CMV vector are restricted by MHC-Ia or an ortholog thereof.

64. The method, use in manufacture, or vector or composition for use of embodiment 63, wherein at least 20%, at least 30%, at least 40%, at least 50%, at least 60%, at least 75%, at least 80%, at least 85%, at least 90%, or at least 95% of the CD8+ T cells elicited by the CMV vector are restricted by MHC-Ia or an ortholog thereof.

65. A method of generating CD4+ T cells that recognize MHC-II/peptide complexes, the method comprising:
(a) administering to a first subject the CMV vector of any one of embodiments 15-34 in an amount effective to generate a set of CD4+ T cells that recognize MHC-II/peptide complexes;
(b) identifying a first CD4+ TCR from the set of CD4+ T cells, wherein the first CD4+ TCR recognizes a MHC-II/fusion protein-derived peptide complex;
(c) isolating one or more CD4+ T cells from a second subject; and
(d) transfecting the one or more CD4+ T cells isolated from the second subject with an expression vector, wherein the expression vector comprises a nucleic acid sequence encoding a second CD4+ TCR and a promoter operably linked to the nucleic acid sequence encoding the second CD4+ TCR, wherein the second CD4+ TCR comprises CDR3α and CDR3β of the first CD4+ TCR, thereby generating one or more CD4+ T cells that recognize MHC-II/peptide complexes.

66. A method of generating CD4+ T cells that recognize MHC-II/peptide complexes, the method comprising:
(a) identifying a first CD4+ TCR from a set of CD4+ T cells, wherein the set of CD4+ T cells are isolated from a subject that has been administered the CMV vector of any one of embodiments 15-34, and wherein the first CD4+ TCR recognizes a MHC-II/fusion protein-derived peptide complex;
(b) isolating one or more CD4+ T cells from a second subject; and
(c) transfecting the one or more CD4+ T cells isolated from the second subject with an expression vector, wherein the expression vector comprises a nucleic acid sequence encoding a second CD4+ TCR and a promoter operably linked to the nucleic acid sequence encoding the second CD4+ TCR, wherein the second CD4+ TCR comprises CDR3α and CDR3β of the first CD4+ TCR, thereby generating one or more TCR-transgenic CD4+ T cells that recognize MHC-II/peptide complexes.

67. A method of generating CD8+ T cells that recognize MHC-Ia/peptide complexes, the method comprising:
(a) administering to a first subject the CMV vector of any one of embodiments 15-34 in an amount effective to generate a set of CD8+ T cells that recognize MHC-Ia/peptide complexes;
(b) identifying a first CD8+ TCR from the set of CD8+ T cells, wherein the first CD8+ TCR recognizes a MHC-Ia/fusion protein-derived peptide complex;
(c) isolating one or more CD8+ T cells from a second subject; and
(d) transfecting the one or more CD8+ T cells isolated from the second subject with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize MHC-Ia/peptide complexes.

68. A method of generating CD8+ T cells that recognize MHC-Ia/peptide complexes, the method comprising:
(a) identifying a first CD8+ TCR from a set of CD8+ T cells, wherein the set of CD8+ T cells are isolated from a subject that has been administered the CMV vector of any one of embodiments 15-34, and wherein the first CD8+ TCR recognizes a MHC-Ia/fusion protein-derived peptide complex;
(b) isolating one or more CD8+ T cells from a second subject; and
(c) transfecting the one or more CD8+ T cells isolated from the second subject with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more TCR-transgenic CD8+ T cells that recognize MHC-Ia/peptide complexes.

69. The method of any one of embodiments 65-68, wherein the first CD4+ TCR or the first CD8+ TCR is identified by DNA or RNA sequencing.

70. The method of any one of embodiments 65-69, wherein the nucleic acid sequence encoding the second CD4+ TCR or the nucleic acid sequence encoding the second CD4+ TCR is identical to the nucleic acid sequence encoding the first CD4+ TCR or the first CD8+ TCR.

71. The method of any one of embodiments 65-70, wherein the first subject is a human.

72. The method of any one of embodiments 65-71, wherein the second subject is a human.

73. A CD4+ T cell generated by the method of any one of embodiments 65, 66, and 69-72.

74. A method of treating or preventing a disease in a subject, the method comprising administering the CD4+ T cell of embodiment 73 to the subject.

75. Use of the CD4+ T cell of embodiment 73 in the manufacture of a medicament for use in treating or preventing a disease in a subject.

76. The CD4+ T cell of embodiment 73 for use in treating or preventing a disease in a subject.

77. A CD8+ T cell generated by the method of any one of embodiments 67-72.

78. A method of treating or preventing a disease in a subject, the method comprising administering the CD8+ T cell of embodiment 77 to the subject.

79. Use of the CD8+ T cell of embodiment 77 in the manufacture of a medicament for use in treating or preventing a disease in a subject.

80. The CD8+ T cell of embodiment 77 for use in treating or preventing a disease in a subject.

### EXAMPLES

### Example 1: Construction of Mtb Antigen Cassettes

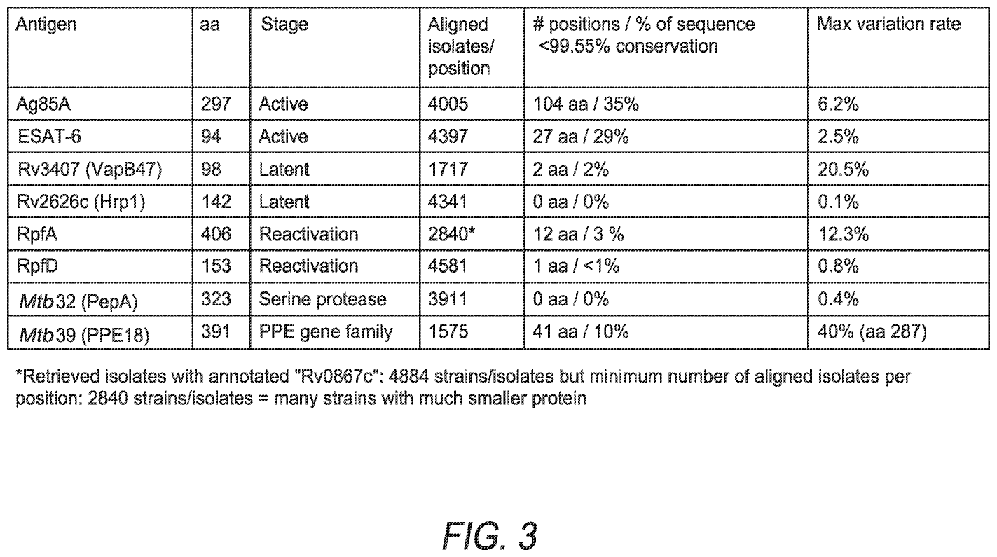

A human CMV vector encoding a fusion protein comprising Mycobacterium tuberculosis (Mtb) antigens was designed. Three Mtb antigen cassettes were evaluated, including Fusion 6, Fusion 7 (Fusion 6 with deletion of RpfA and insertion of a variant of the fusion protein M72, “M72-fusion-2”, at the RpfA site), and Fusion 8 (Fusion 6 plus addition of M72-fusion-2 at the C-terminal) (FIG. 2). The variant of M72, “M72-fusion-2”, included in Fusion 7 and Fusion 8 is M72 wherein the N-terminal two-residue histidine tag and the methionine at amino acid position 1 have both been removed (SEQ ID NO.: 22). A table summarizing conservation of Mtb antigens and RpfA variants is shown in FIG. 3.

Components of Fusion 6 Fusion 6 is a Mtb fusion protein comprising Ag85A, ESAT6, Rv3407, Rv2626c, RpfA, and RpfD.

### Design of Fusion 7

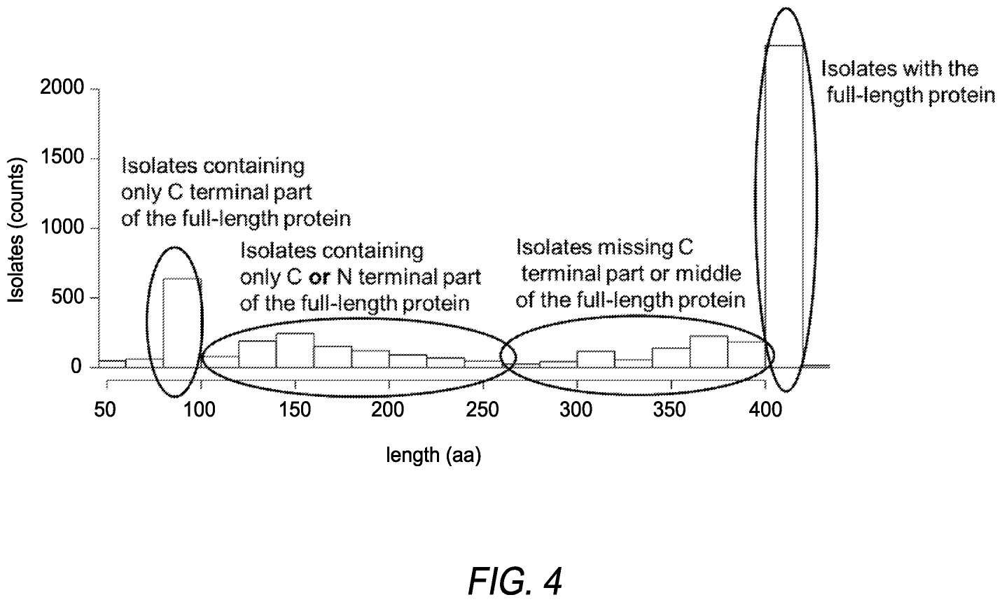

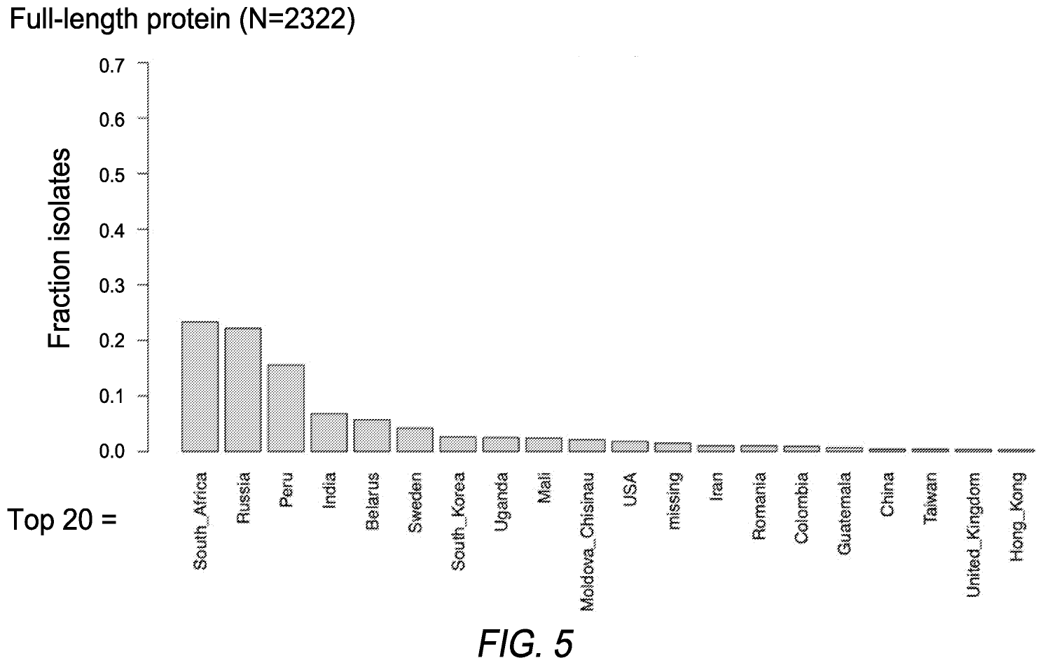

Fusion 7 was constructed based on Fusion 6. To create Fusion 7, RpfA was first deleted. RpfA has variable expression in Mtb strains (FIG. 4). For example, RpfA in many Mtb strains contains only the C-terminus (starting at 321 bp in RpfA) of the isoform of Fusion 6. Additionally, a subset of Mtb strains have only the RpfA N-terminus and others lack both the N-terminus and the middle portion. Mtb isolates with the shortest RpfA (C-terminus only; <100 amino acids) are predominantly from the UK and the Netherlands. Mtb isolates from South Africa have variable RpfAs and may include the C-terminus only, the N-terminus only, or full length RpfA (FIG. 5). Next, M72-fusion-2 was inserted into the RpfA site. M72-fusion-2 is derived from, M72, a fusion protein derived from two M. tuberculosis antigens, Mtb32a and TbH9, that functions as a vaccine antigen. Mtb32a is a serine protease conserved in virulent and avirulent Mtb strains. TbH9 (also known as Mtb39a) is a PPE/PE family protein encoded by Rv1196/ppe18. PPE/PE proteins are known to be prominent targets of Mtb immunity. TbH9 is among antigens identified by TCR profiling of “controllers” versus “progressors” in Mtb. RpfA was deleted to accommodate expression of the M72 antigens while maintaining a genetically stable vector. TbH9 is a CD4 and CD8 T cell target in latency. The final Fusion 7 construct (SEQ ID NO: 42) comprises Ag85A, ESAT6-Rv3407-Rv2626c, M72-fusion-2 (Mtb32A and TbH9), and RpfD.

### Design of Fusion 8

Fusion 8 was constructed based on Fusion 6, similarly to the construction of Fusion 7 except that M72-fusion-2 was added to the C-terminal end of Fusion 6 and RpfA was not deleted.

### Example 2: Evaluation of Mtb Antigen Cassettes for Antigen Expression, Genetic Stability, and Immunogenicity

Mtb antigen cassettes were paired with either a UL82 or UL78 promoter. Antigen expression, genetic stability, and immunogenicity (in rhesus macaques) was evaluated.

TABLE 1
Mtb antigen cassette and promoter combinations for evaluation.

| Mtb Antigen Cassette | Promoter |
|---|---|
| Fusion 6 | UL78 promoter |
| Fusion 7 | UL78 promoter |
| Fusion 8 | UL78 promoter |
| Fusion 6 | UL82 promoter |
| Fusion 7 | UL82 promoter |
| Fusion 8 | UL82 promoter |

Antigen expression for six antigen cassette and promoter combinations (Table 1) was tested in bacterial artificial chromosomes (BACs) by western blot with ESAT-6 antibody. All six BACs had Mtb antigen expression with bands in the expected size range.

Genetic stability was evaluated using the same BACs. Three clones per construct were passaged to mimic the drug production process (Research Seed Stock with three additional passages (RSS+3), for a total of four passages) with next-generation sequencing (NGS) at each passage. Passages were performed in T-150 flasks at an MOI of 0.003 for UL78-containing constructs or at an MOI of 0.01 for UL82-containing constructs. Genetic stability was defined as observing no significant modifications that increase with passage (i.e., deletions and/or rearrangements involving the transgene or U_{L}/1b′ region). BACs expressing Fusion 6 +UL78 promoter and Fusion 6 +UL82 promoter were found to be stable through RSS+3. The BAC expressing Fusion 8 +UL78 promoter showed insert and vector backbone instability. BACs expressing Fusion 7 +UL78 promoter and Fusion 7 +UL82 promoter were stable through RSS+2 and RSS+3. The BAC expressing Fusion 8 +UL82 promoter was stable through RSS+2 and RSS+3.

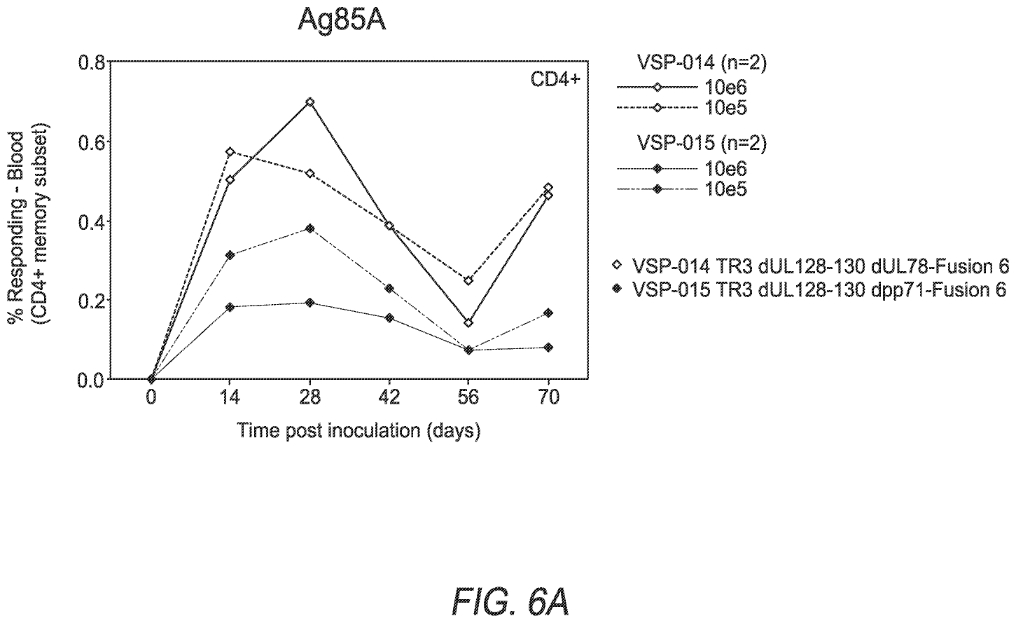

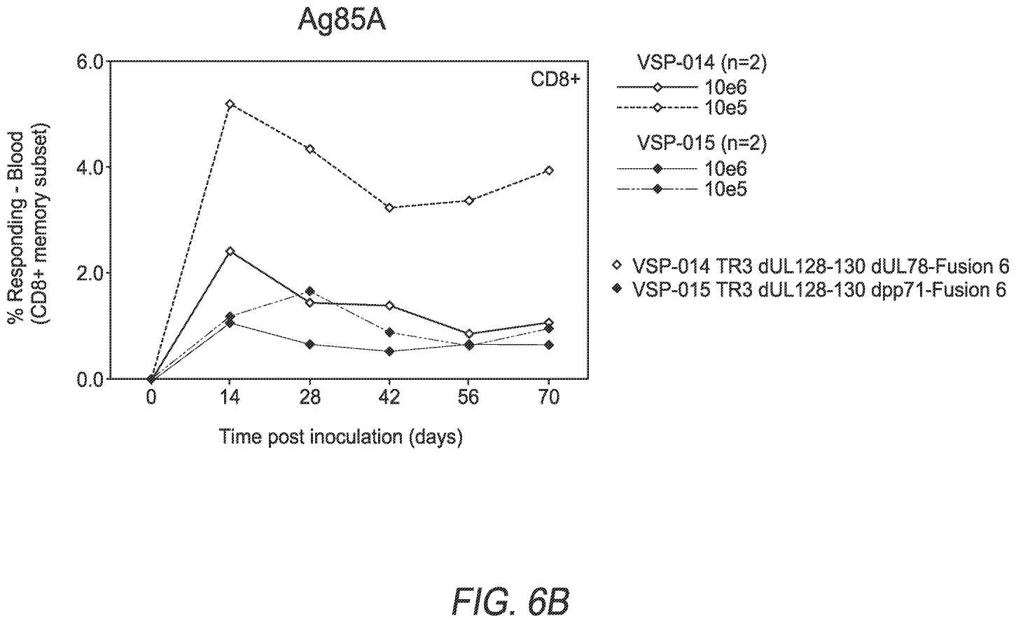

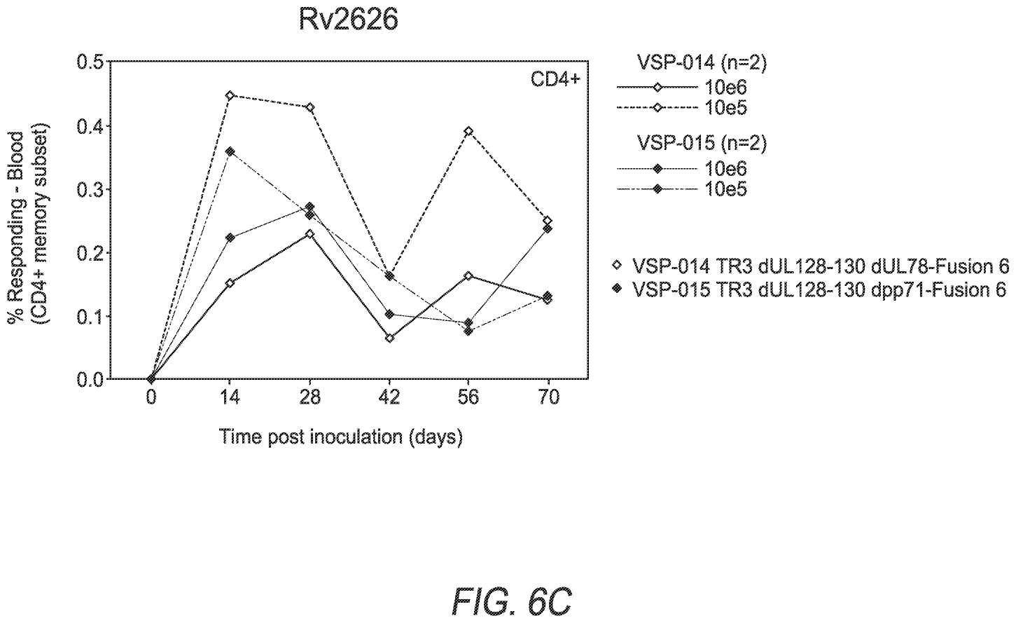

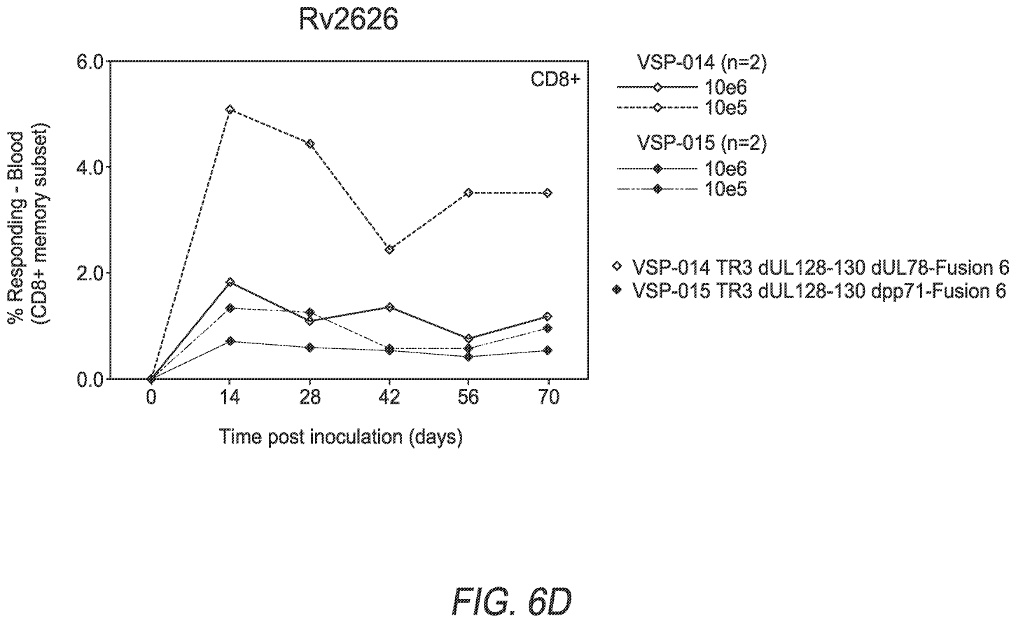

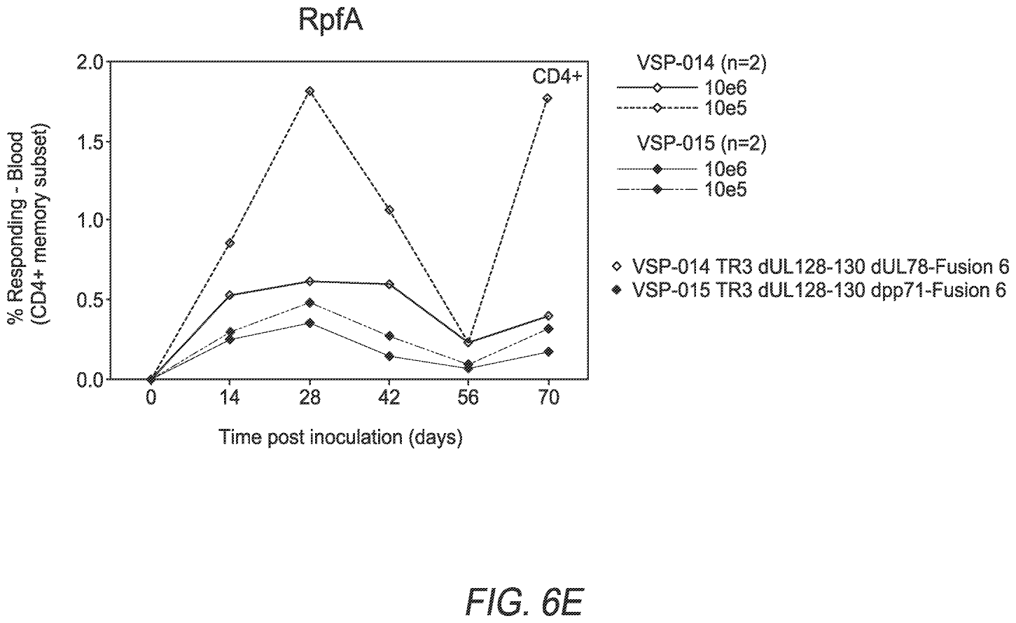

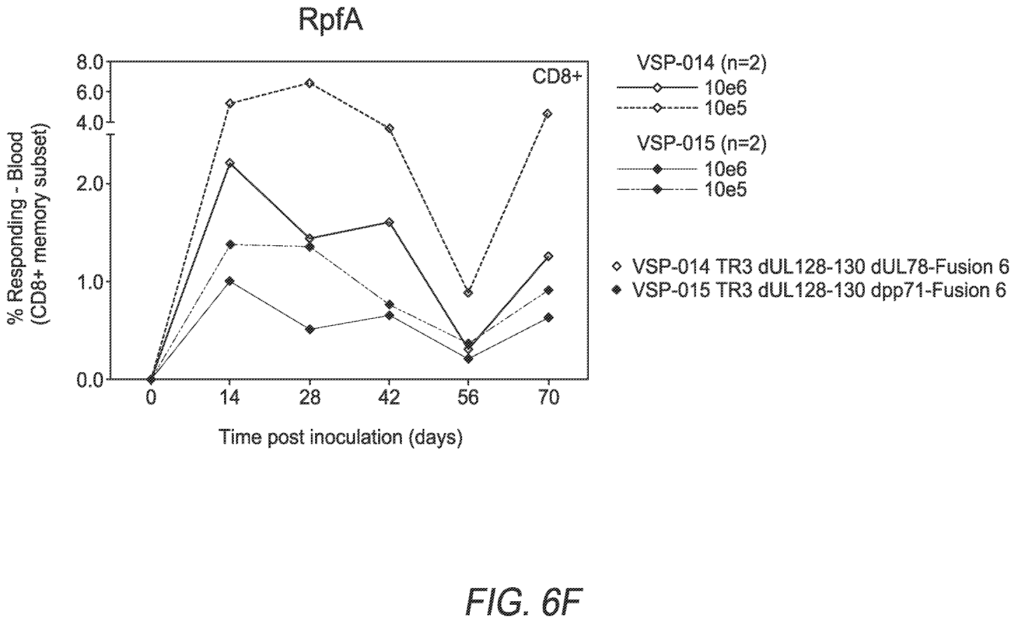

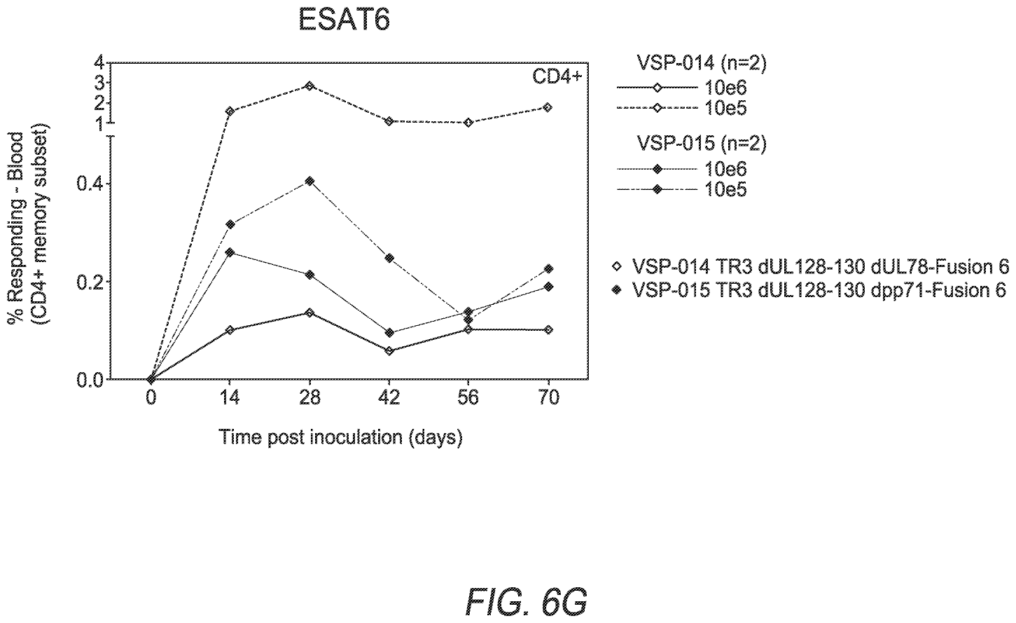

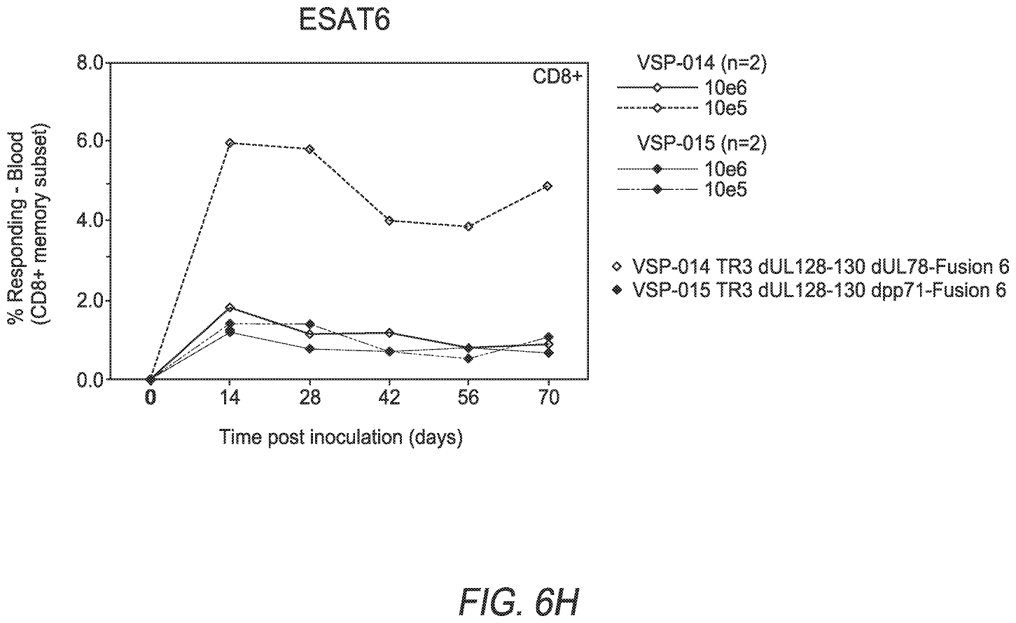

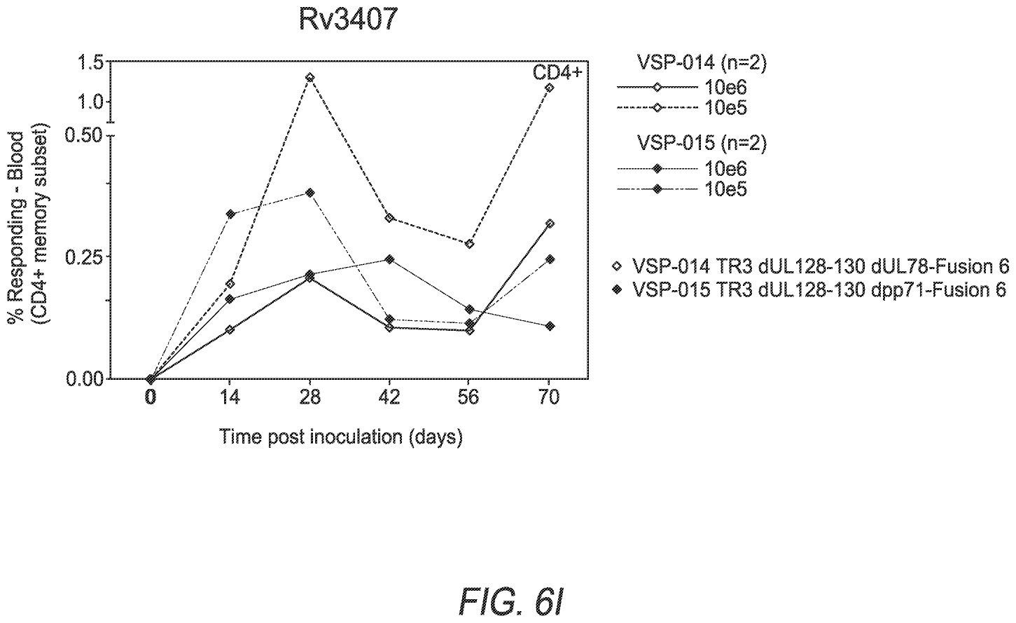

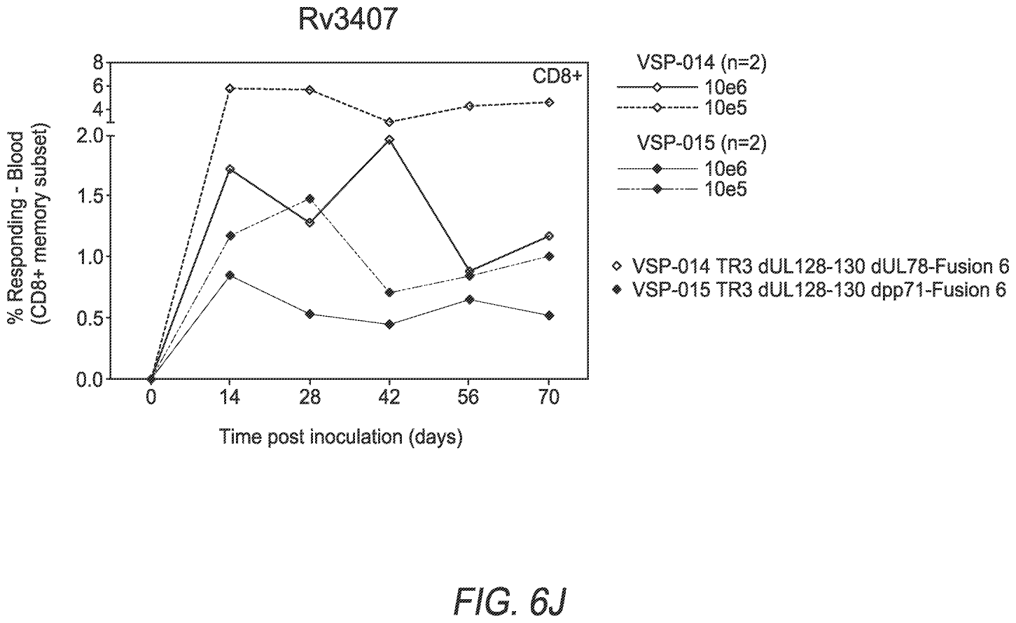

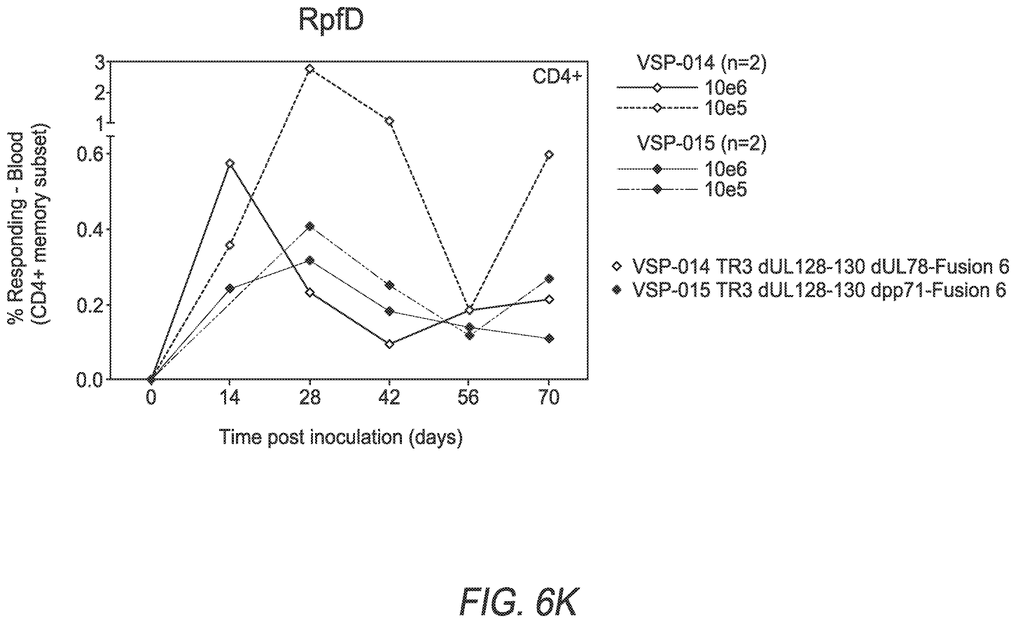

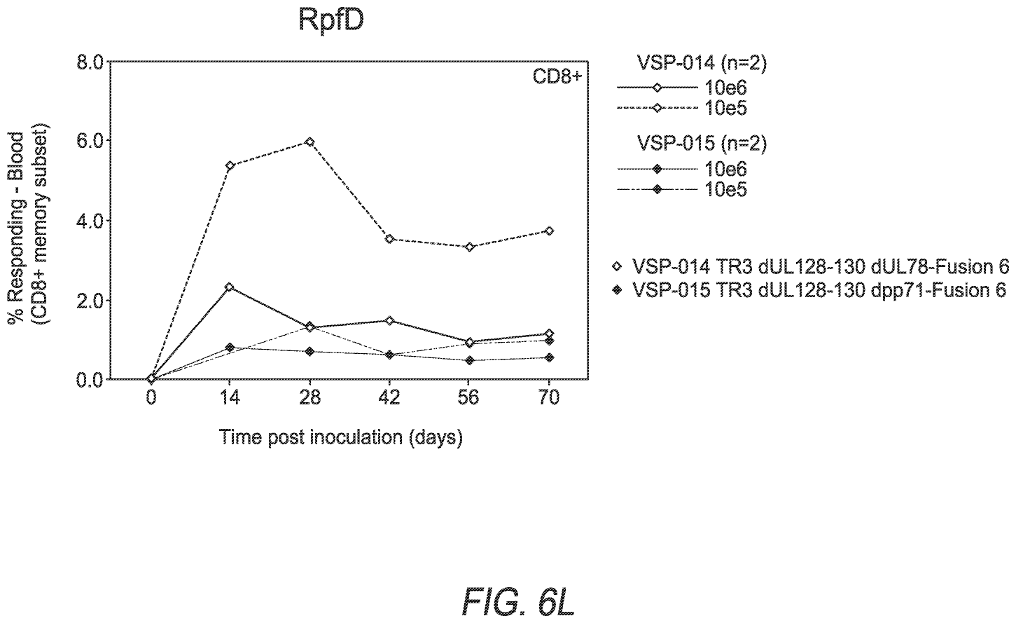

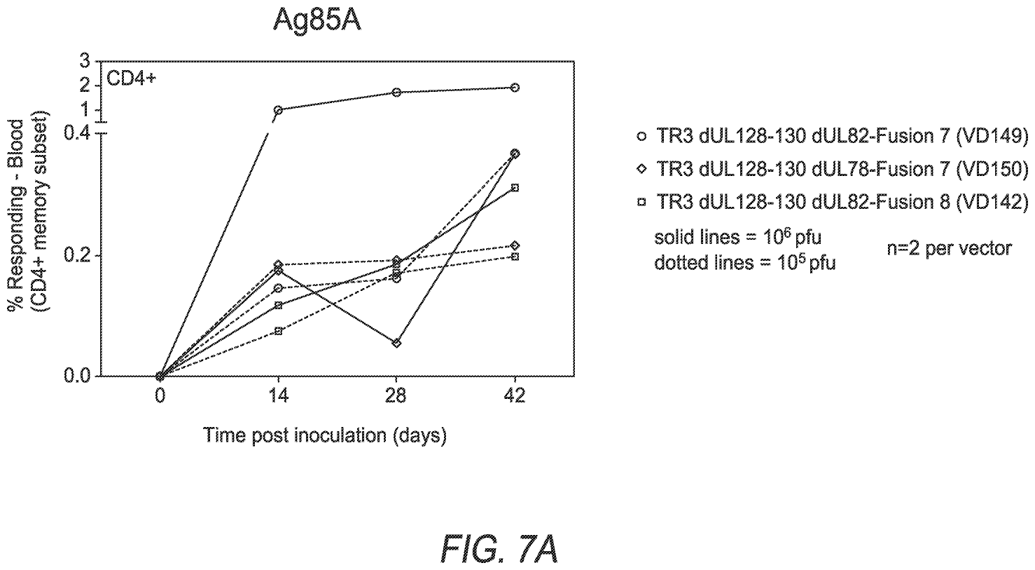

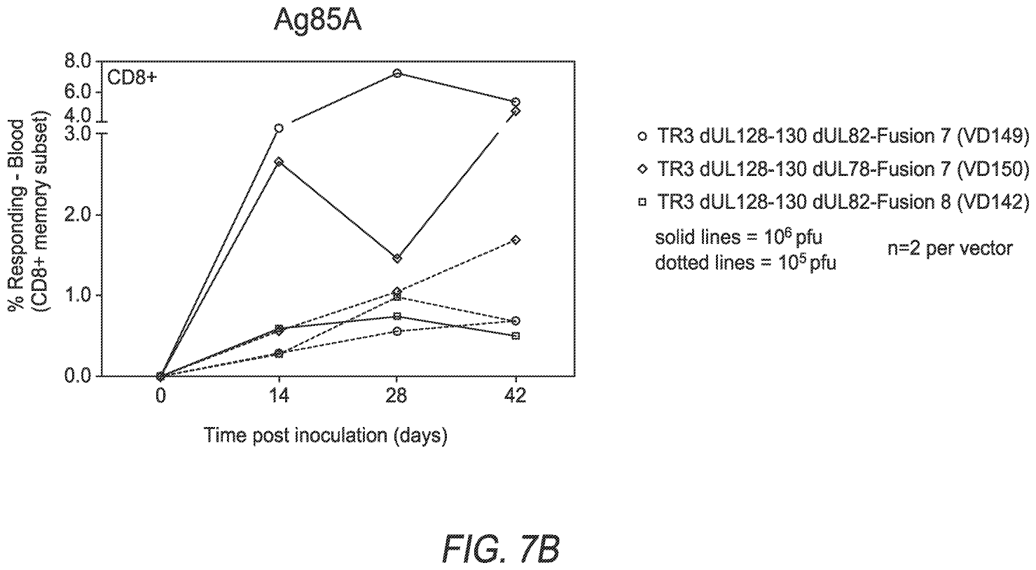

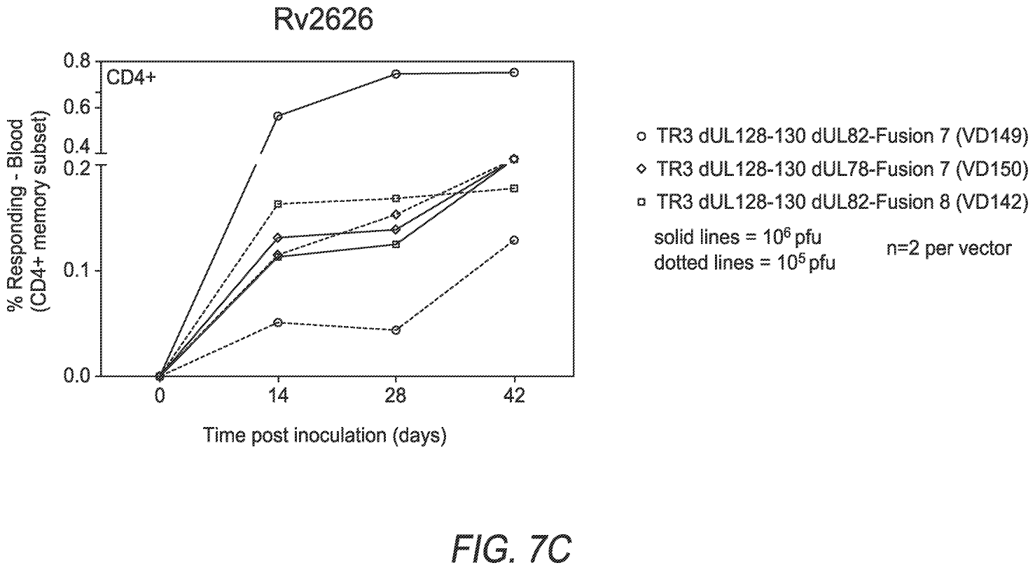

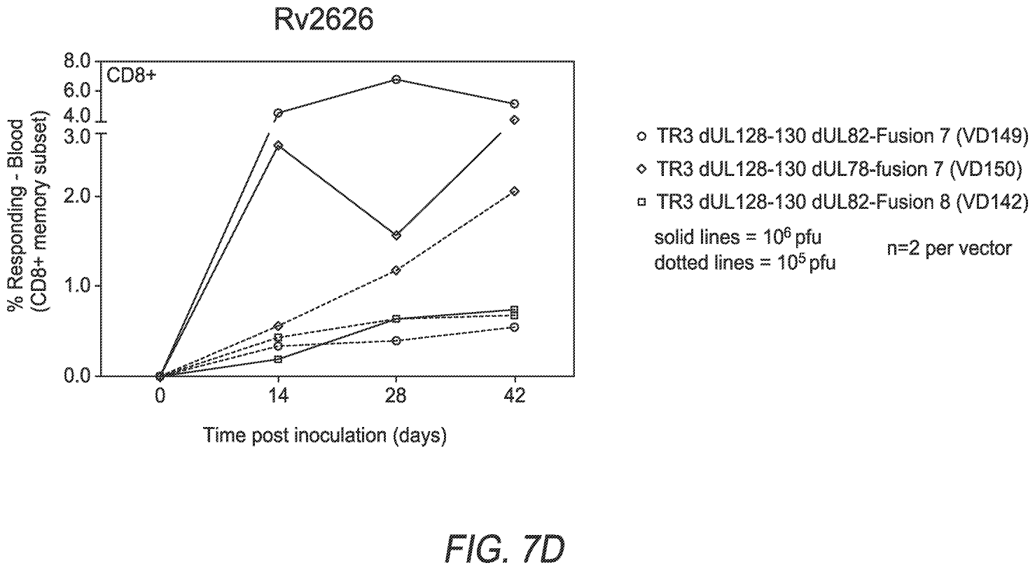

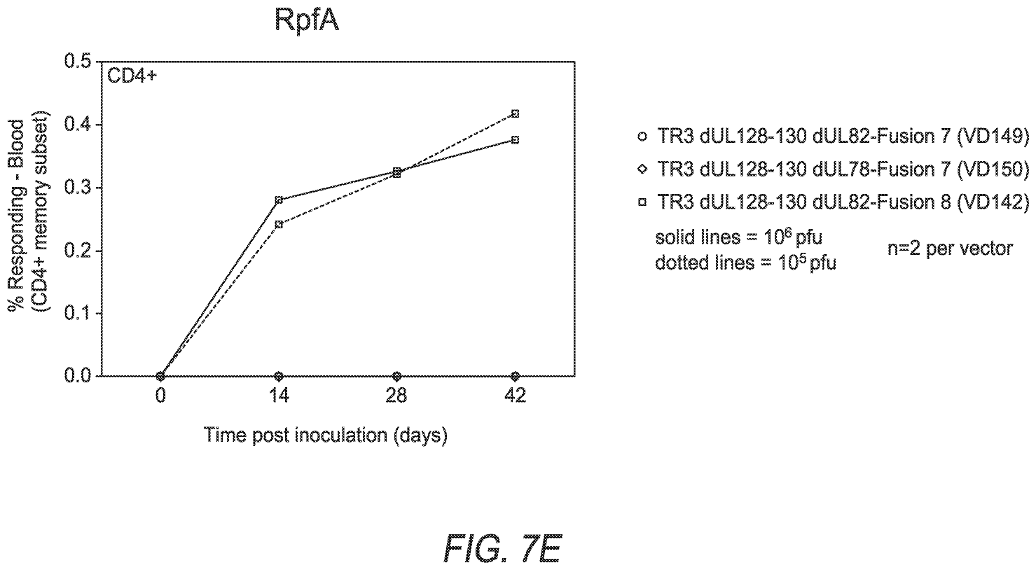

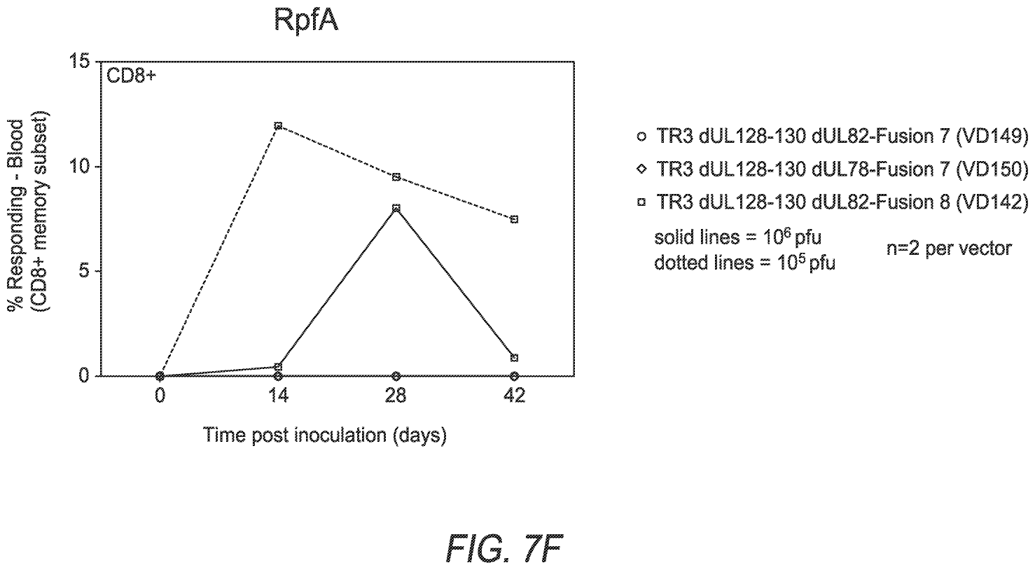

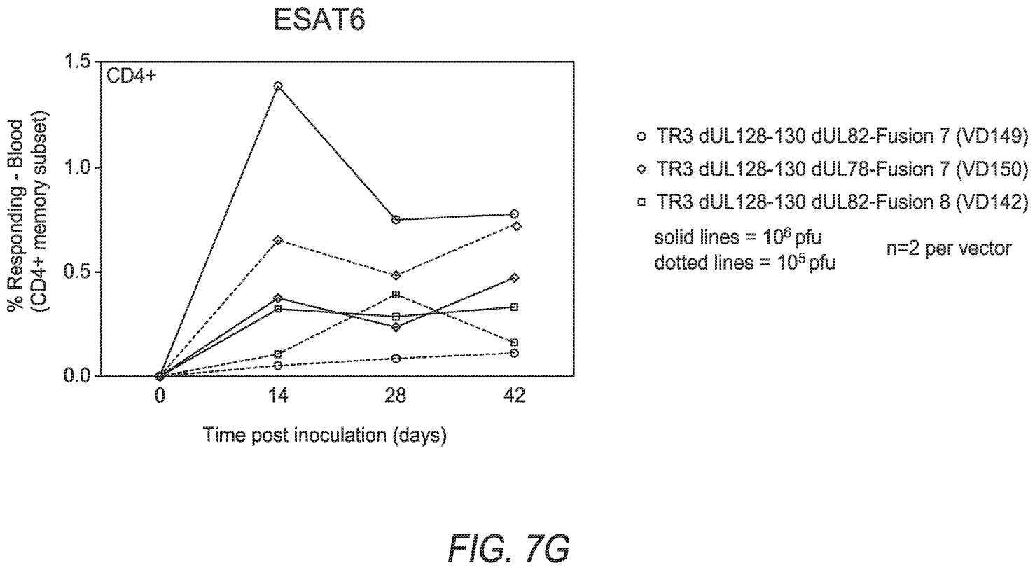

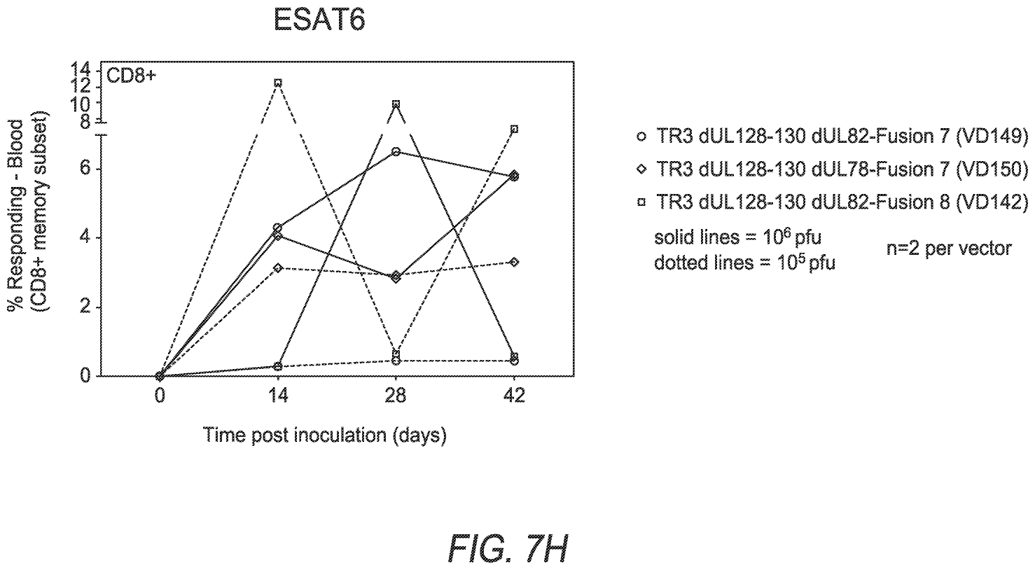

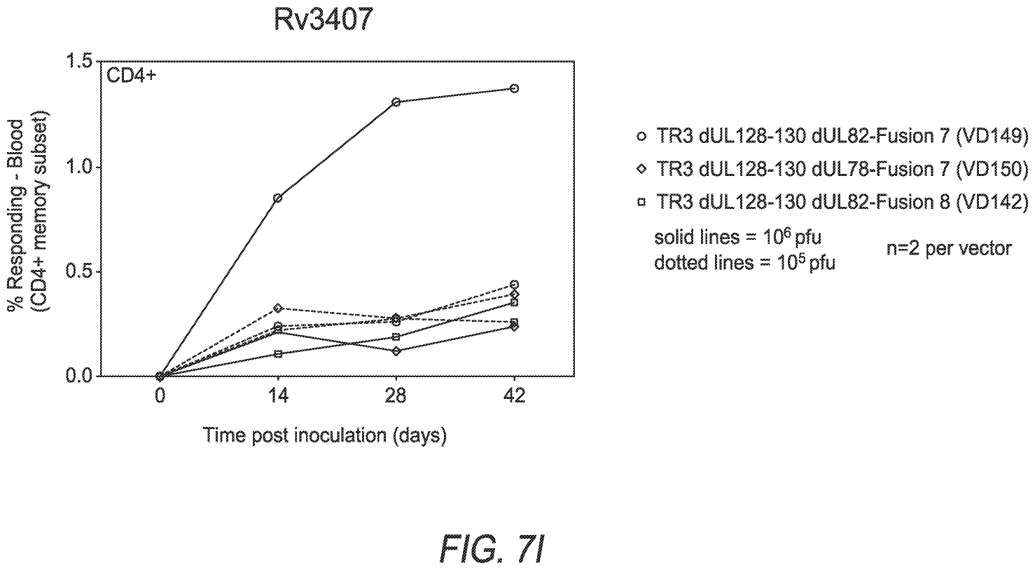

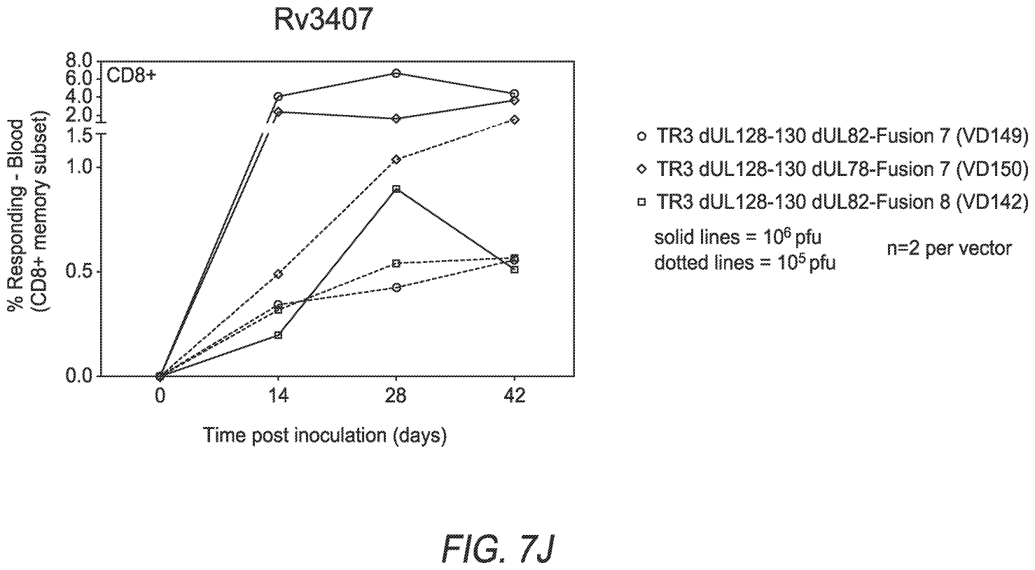

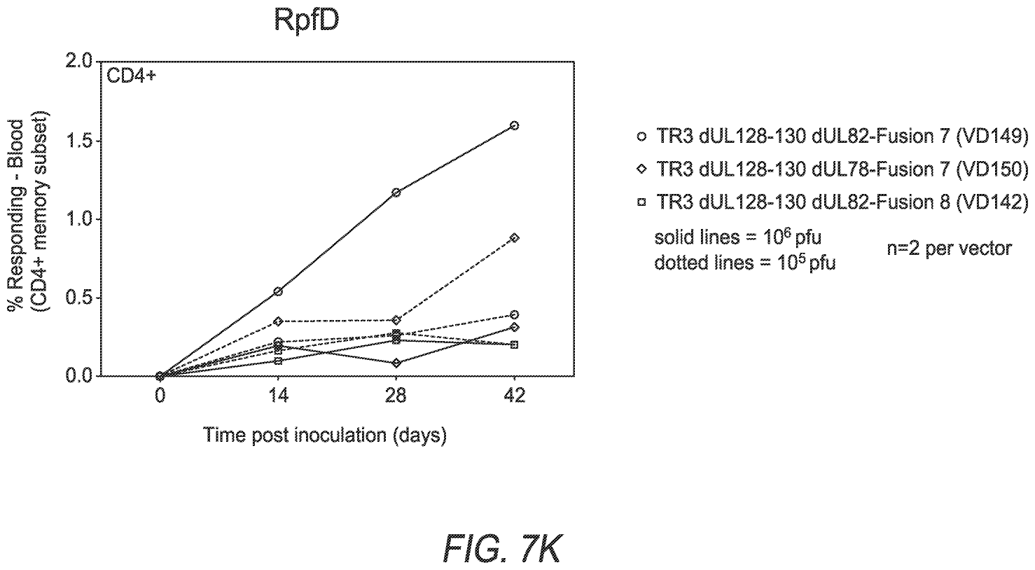

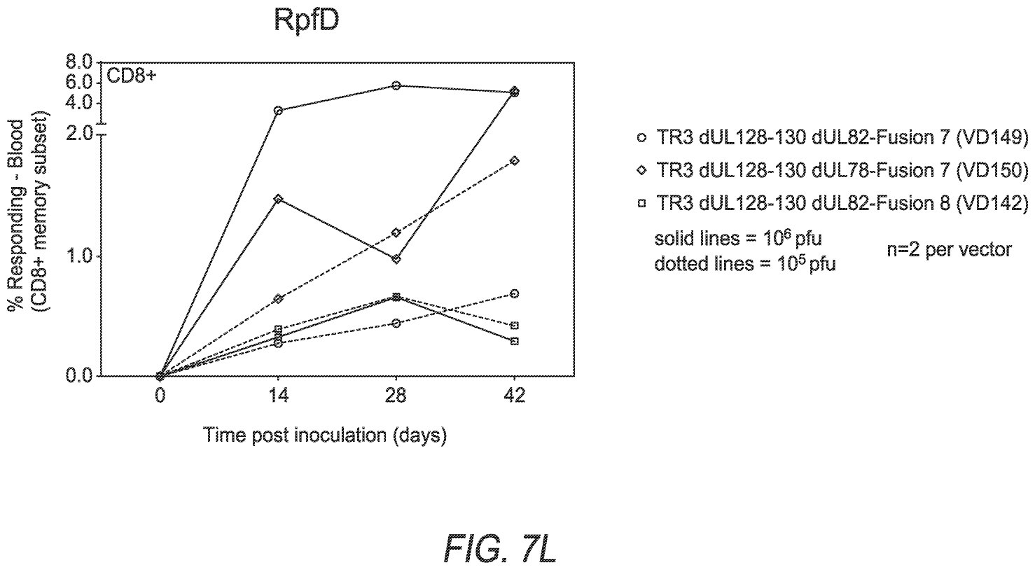

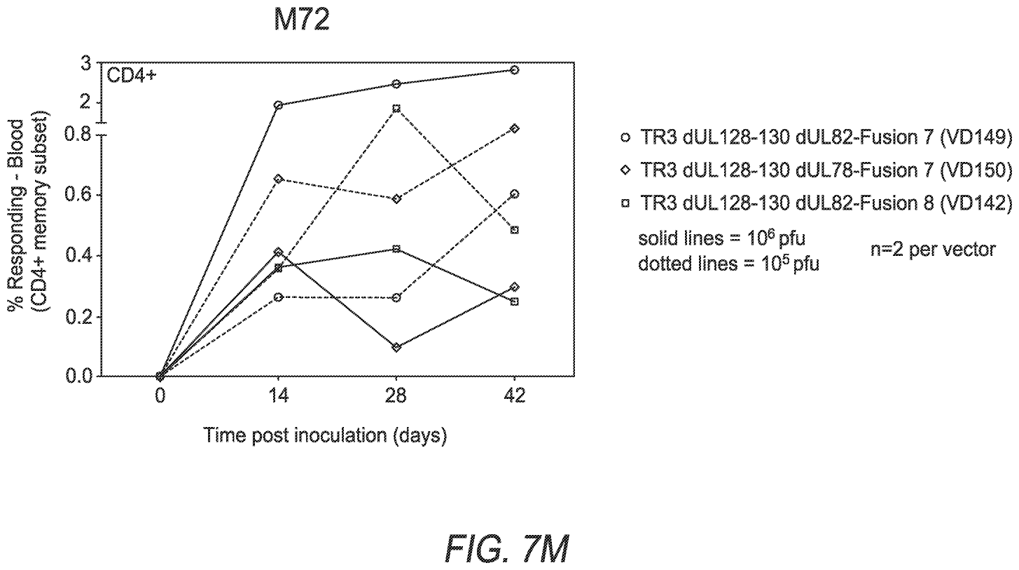

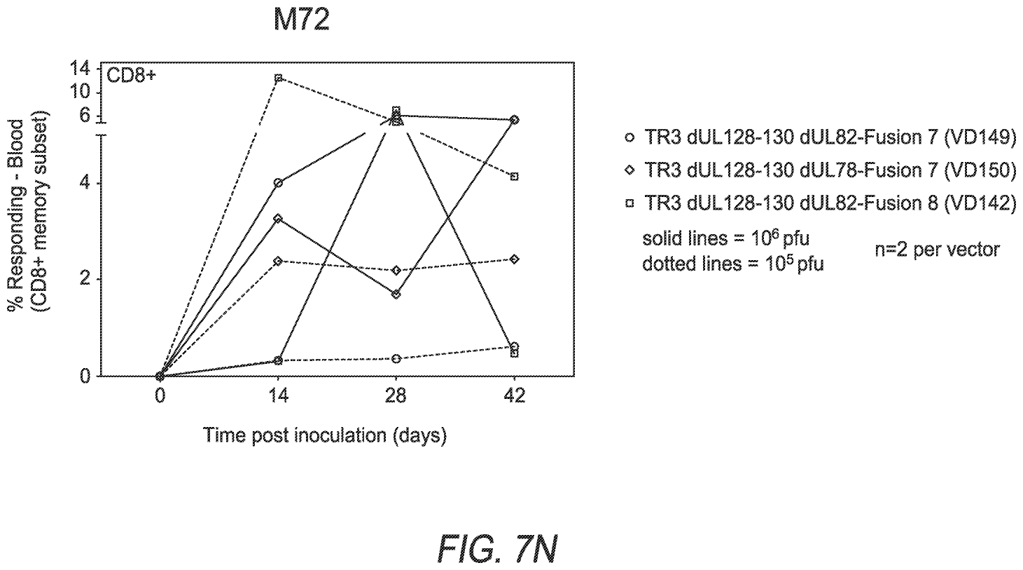

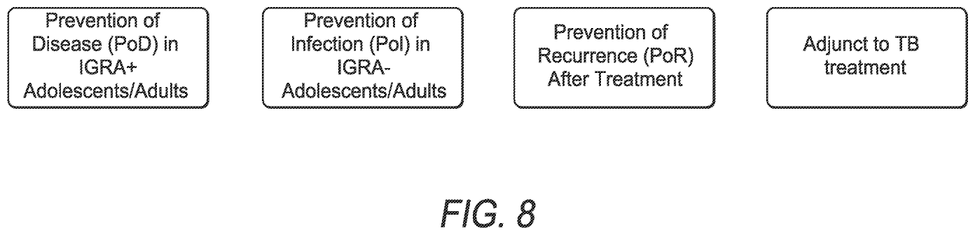

Immunogenicity was evaluated in a rhesus macaque model. Rhesus macaques were administered HCMV viral vectors expressing six antigen cassette and promoter combinations (Table 1) at a dose of 10⁶ pfu. Peripheral blood mononuclear cells (PBMCs) were isolated in two week increments for up to ten weeks, then stimulated with Mtb peptide pools containing peptides from genes expressed in Fusion 6, or Fusion 6 and M72 prior to intracellular cytokine staining (ICS). Frequencies of CD4+ and CD8+ T cells, frequencies of IFNγ+ and/or TNFα+ cells, and memory/effector T cell phenotypes were determined. In animals administered viral vectors expressing Fusion 6 +UL78 promoter or Fusion 6 +UL82 promoter, CD4+ and CD8+ T cell responses to Fusion 6 peptides were detected at 6 weeks post-dosing (FIGS. 6A-6L). In animals administered viral vectors expressing Fusion 7 +UL78 promoter, Fusion 7 +UL82 promoter, or Fusion 8 +UL82 promoter, T cell responses to peptide pools were detected at 6 weeks (FIGS. 7A-7N). T cell response was indicated by the detection of IFNγ+ and/or TNFα+CD4+ and/or CD8+ T cells.

All three Mtb antigen designs were functional. The Fusion 7 antigen cassette elicited CD4+ and CD8+ T cell responses in rhesus macaques. RpfA has variable expression in Mtb strains, justifying replacement with M72-fusion-2, which confers protection against CMV in human studies. The Fusion 6 antigen cassette incorporates a broad range of Mtb antigens incorporated from different stages of the infectious cycle (active/latent/reactivation stages) and has been shown to be protective in rhesus macaque studies. The Fusion 8 antigen cassette includes the Fusion 6 cassette as well M72-fusion-2, but can be associated with genetic instability.

### Example 3: Selection of a hCMV-TB Vector Backbone and TB-Antigen Promoter

### Vector Backbone Selection

Mtb-specific CD4+ T cells are considered more important for infection control than CD8+ T cells. A non-human primate study of intravenous BCG (Bacillus Calmette-Guerin) vaccination suggests the importance of antigen-specific T cell frequency. Additionally, the GSK M72-ASO1E vaccine elicits antibodies and CD4+ T cells but few or no CD8+ T cells (Penn-Nicholson A. et al., Safety and immunogenicity of candidate vaccine M72/ASO1E in adolescents in a Mtb endemic setting. Vaccine. 2015 Jul. 31; 33(32):4025-34). Thus, eliciting Mtb-specific CD4+ T cells is important for CMV-TB vaccine design and is preferred to eliciting CD8+ T cells at the expense of the CD4+ T cell response. Further, eliciting conventional class I-restricted CD8+ T cells is preferable to eliciting to class II/MHC-E restricted CD8+ T cells as class II/MHC-restriction was shown to not be required for Rh-CMV Mtb protection.

The CMV vector backbone described here is known to elicit a robust CD4+ T cell response. Additionally, the backbone contains an intact UL146-147, which is expected to induce a Class I restricted CD8+ T cell response. The backbone comprises deletions of UL128-130, which is known to promote genetic stability in MRC-5 fibroblast cells.

A CMV-TB vector was constructed with features as shown in Table 2.

TABLE 2
Features of CMV-TB vector

| Function | Feature |
|---|---|
| Tropism & Immune | ΔUL128/UL130 |
| programing | UL146/147 intact |
| | UL18 intact |
| | ΔUL78 or ΔUL82 |
| Growth restriction | ΔUL82 or ΔUL78 |
| Antigen delivery | Mtb Antigen |
| | UL82 or UL78 |

### Mtb Antigen Cassette Promoter Selection

A Mtb fusion protein can be inserted into the CMV vector backbone to replace a gene (e.g., UL82 or UL78) such that the fusion protein is operably linked to and is expressed by the promoter of the deleted gene. The UL78 promoter was found to effectively drive expression of Fusion 7 or Fusion 6 and the UL82 promoter was found to effectively drive expression of Fusion 8. Deletion of the HCMV gene UL82 (which encodes the tegument protein pp71) is known to create a growth deficiency, resulting in lower viral yield. Additionally, expression of exogenous pp71 is also known to increase transgene expression from UL82-deleted CMV vectors (Caposio P. et al., Characterization of a live-attenuated HCMV-based vaccine platform. Sci Rep. 2019 Dec. 17; 9(1):19236). Therefore, use of a UL82 promoter will require exogenous pp71 expression in order to achieve the highest levels of transgene expression and viral yield. In contrast, use of a UL78 promoter eliminates the need for exogenous expression of pp71 mRNA during production.

### Example 4: A Phase 1A/1B Study to Evaluate the Safety, Reactogenicity, Tolerability, and Immunogenicity of Vector 4

A Phase 1a/1b study has been designed to evaluate the safety, reactogenicity, tolerability, and immunogenicity of Vector 4 (SEQ ID NO:44). Four groups of patients will be evaluated based on CMV status (positive or negative), interferon-7 release assay result (positive or negative), and receipt of prior BCG (bacille Calmette-Guerin) vaccination (positive or negative). “Part A” will include CMV+/IGRA− subjects, “Part B” will include CMV−/IGRA− subjects, “Part C” will include CMV+/IGRA−/BCG+ subjects, and “Part D” will include CMV+/IGRA+/BCG+ subjects. The study will take place across multiple sites in the United States (Part A and Part B groups) and one to two sites in countries where Mtb is endemic (e.g. South Africa). Each cohort will be composed of ten (10) patients, with eight (8) receiving the drug, and two (2) receiving placebo.

### Example 5: A Development Plan to Evaluate Vector 4 for Use in Multiple Tuberculosis-Related Indications

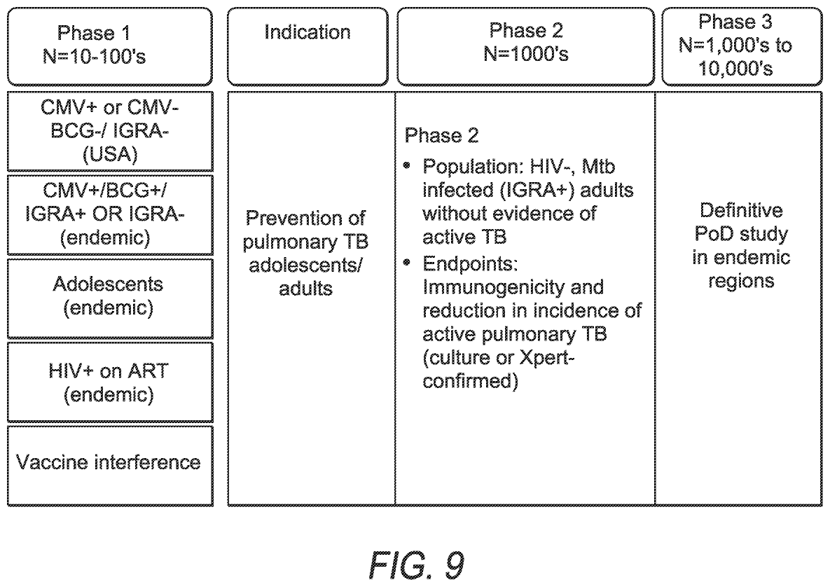

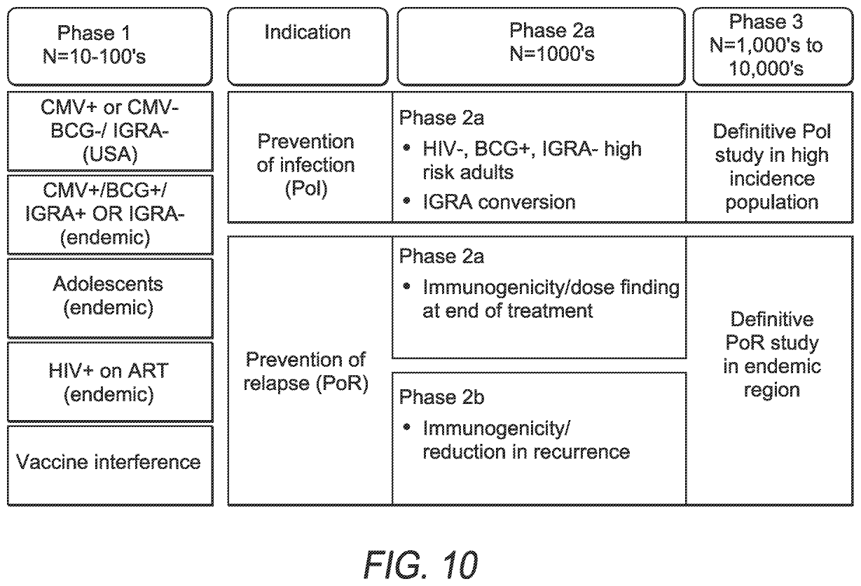

FIG. 9 shows a development plan to evaluate Vector 4 (SEQ ID NO:44) for use in prevention of pulmonary tuberculosis in adolescents and adults. FIG. 10 shows a development plan to evaluate Vector 4 (SEQ ID NO:44) for use in prevention of M. tuberculosis infection and prevention of tuberculosis relapse in adolescents and adults.

While specific embodiments have been illustrated and described, it will be readily appreciated that the various embodiments described above can be combined to provide further embodiments, and the various embodiments described above can be combined to provide further embodiments.

All of the U.S. patents, U.S. patent application publications, U.S. patent applications, foreign patents, foreign patent applications, and non-patent publications referred to in this specification and/or listed in the Application Data Sheet, including U.S. Provisional Application Nos. 63/239,278 filed on Aug. 31, 2021 and 63/392,778 filed on Jul. 27, 2022 are incorporated herein by reference, in their entirety, unless explicitly stated otherwise. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A fusion protein comprising or consisting of
Ag85A-ESAT-6-Rv3407-Rv2626c-Ra12-TbH9-Ra35-RpfD.

2. A fusion protein encoded by a nucleic acid comprising a sequence having 90%, 91%, 92%, 93%, 94%, 95%, 96%, 97%, 98%, 99%, or 10000 identity to the nucleic acid sequence according to SEQ TD NO:41.

3. (canceled)

4. The fusion protein of claim 1, further comprising a poly-His tag, wherein optionally,
(i) the poly-His tag comprises or consists of two to six His residues;
(ii) the poly-His tag is located at the N-terminus of the fusion protein; and/or
(iii) the poly-His tag is inserted after the initial Met residue.

5.-7. (canceled)

8. The fusion protein of claim 1, further comprising a HA tag, wherein optionally, the HA tag is located at the C-terminus of the fusion protein.

9. (canceled)

10. The fusion protein of claim 1, wherein the fusion protein further comprises one or more linkers connecting one or more of Ag85A, ESAT-6, Rv3407, Rv2626c, RpfA, RpfD, Ra12, TbH9, and Ra35, wherein each of the one or more linkers comprises or consists of one or more amino acid residues.

11. A nucleic acid molecule encoding the fusion protein according to claim 1.

12. A vector comprising the nucleic acid molecule of claim 11.

13. The vector of claim 12, further comprising a promoter, wherein a promoter is operably linked to the nucleic acid molecule encoding the fusion protein.

14. (canceled)

15. The vector of claim 12, wherein the vector is a cytomegalovirus (CMV) vector.

16.-19. (canceled)

20. The vector of claim 15, wherein the viral vector is a RhCMV vector, a HCMV vector, or a recombinant HCMV vector.

21. The vector of claim 15, wherein the promoter is operably linked to the nucleic acid molecule encoding the fusion protein and the promoter is a UL78 promoter, or an ortholog thereof, wherein optionally, the nucleic acid molecule encoding the fusion protein replaces all or part of UL78.

22.-26. (canceled)

27. A recombinant HCMV vector comprising a TR3 backbone and a nucleic acid sequence encoding a heterologous antigen according to SEQ ID NO: 42, wherein:
(a) the vector does not express UL128 or UL130, or orthologs thereof,
(b) the vector comprises a nucleic acid sequence encoding UL146, UL147, UL18, and UL82 or orthologs thereof, and
(c) the heterologous antigen replaces all or part of UL78 and is operably linked to the UL78 promoter.

28. The recombinant HCMV vector of claim 27, wherein the recombinant HCMV vector comprises a nucleic acid sequence having at least 90%, 91%, 92%, 93%, 94%, 95%, 96%, 97%, 98%, 99%, or 100% identity to the nucleic acid sequence according to SEQ ID NO:44.

29. The vector of claim 15, wherein the RhCMV or HCMV vector (i) comprises a nucleic acid sequence encoding UL146 and a nucleic acid sequence encoding UL147, or orthologs thereof, and (ii) does not express UL128 or UL130, or orthologs thereof.

30. The vector of claim 27, wherein the vector does not express a UL128 protein or a UL130 protein, resulting from the presence of one or more mutations in the nucleic acid sequences encoding UL128 and UL130.

31. The vector of claim 30, wherein the mutation in the nucleic acid sequences encoding UL128 and UL130 is a point mutation, frameshift mutation, truncation mutation, or deletion of all of the nucleic acid sequences encoding the viral protein.

32. The vector of claim 15, wherein the vector is a HCMV vector comprising a TR3 backbone.

33. The vector of claim 15, wherein the vector further comprises a nucleic acid sequence encoding a microRNA (miRNA) recognition element (MRE), wherein the MRE contains a target site for a miRNA expressed in endothelial cells and/or a miRNA expressed in myeloid cells.

34. (canceled)

35. A pharmaceutical or immunogenic composition comprising the vector of claim 12 and a pharmaceutically acceptable carrier.

36. (canceled)

37. A method of generating an immune response in a subject, comprising administering to the subject the vector of claim 12.

38.-39. (canceled)

40. A method of treating or preventing tuberculosis in a subject, comprising administering to the subject the vector of claim 12.

41.-42. (canceled)

43. The method of claim 40, wherein the subject is CMV positive.

44.-50. (canceled)

51. The method of claim 40, wherein the tuberculosis is a latent, pulmonary, and/or recurrent tuberculosis infection.

52.-59. (canceled)

60. A method of generating CD4+ T cells that recognize MHC-II/peptide complexes, the method comprising:
(a) administering to a first subject the CMV vector of claim 15 in an amount effective to generate a set of CD4+ T cells that recognize MHC-II/peptide complexes;
(b) identifying a first CD4+ TCR from the set of CD4+ T cells, wherein the first CD4+ TCR recognizes a MHC-II/fusion protein-derived peptide complex;
(c) isolating one or more CD4+ T cells from a second subject; and
(d) transfecting the one or more CD4+ T cells isolated from the second subject with an expression vector, wherein the expression vector comprises a nucleic acid sequence encoding a second CD4+ TCR and a promoter operably linked to the nucleic acid sequence encoding the second CD4+ TCR, wherein the second CD4+ TCR comprises CDR3α and CDR3β of the first CD4+ TCR, thereby generating one or more CD4+ T cells that recognize MHC-II/peptide complexes.

61. (canceled)

62. A method of generating CD8+ T cells that recognize MHC-Ia/peptide complexes, the method comprising:
(a) administering to a first subject the CMV vector of claim 15 in an amount effective to generate a set of CD8+ T cells that recognize MHC-Ia/peptide complexes;
(b) identifying a first CD8+ TCR from the set of CD8+ T cells, wherein the first CD8+ TCR recognizes a MHC-Ia/fusion protein-derived peptide complex;
(c) isolating one or more CD8+ T cells from a second subject; and
(d) transfecting the one or more CD8+ T cells isolated from the second subject with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize MHC-Ia/peptide complexes.

63.-67. (canceled)

68. A CD4+ T cell generated by the method of claim 60.

69. A method of treating or preventing a disease in a subject, the method comprising administering the CD4+ T cell of claim 68 to the subject.

70.-71. (canceled)

72. A CD8+ T cell generated by the method of claim 62.

73. A method of treating or preventing a disease in a subject, the method comprising administering the CD8+ T cell of claim 72 to the subject.

74.-75. (canceled)
